(19) United States
(12) Patent Application Publication
(10) Pub. No.: US 2012/0141549 A1
(43) Pub. Date: Jun. 7, 2012

(54) OLIGONUCLEOTIDES TARGETING ASTHMA INFLAMMATION PROCESSES

(76) Inventors: Nick Giannoukakis, Coraopolis, PA (US); Massimo Trucco, Pittsburgh, PA (US); Wilson Meng, Sewickley, PA (US)

(21) Appl. No.: 13/280,777

(22) Filed: Oct. 25, 2011

Related U.S. Application Data
(60) Provisional application No. 61/406,409, filed on Oct. 25, 2010.

(51) Int. Cl. A61K 9/14; A61P 11/06; A61P 29/00; A61P 11/00; A61K 31/7088; A61K 31/711

(52) U.S. Cl. 424/400; 514/44 A

(57) ABSTRACT

The present invention relates to methods and compositions for inhibiting inflammatory processes (such as asthma) in the lungs, wherein oligonucleotides targeting IgE receptors and NfkappaB are administered via an aerosolized microsphere formulation.

### PRIORITY

This application claims priority to U.S. Provisional Application No. 61/406,409, filed Oct. 25, 2010, the contents of which is hereby incorporated by reference in its entirety herein.

### GRANT INFORMATION

Not applicable.

### 1. INTRODUCTION

The present invention relates to methods and compositions for inhibiting inflammatory processes (such as asthma) in the lungs, wherein oligonucleotides targeting IgE receptors and NFkappaB are administered via an aerosolized microsphere formulation.

### 2. BACKGROUND OF THE INVENTION

Allergic asthma is a complex inflammatory disease of the lungs characterized by variable airflow obstruction, airway hyperresponsiveness (AHR) and airway inflammation (1-7). The inflammatory process consists of a chronic infiltration by mast cells, basophils, eosinophils, dendritic cells and B/T-lymphocytes, each playing a distinct role as part of a network of local inflammation (2, 4, 6-12).

The key biomarker of allergic asthma is the IgE antibody, now considered to be the critical “gatekeeper” of the human allergic response (4, 10, 13-23). Mast cells and basophils are the main cellular modulators of IgE bioactivity and largely responsible for the generation of the autacoids that amplify the asthmatic phenotype in lungs (1, 6, 7, 10, 24-30). These autacoids affect not only the recruitment and function of other immune cells in the vicinity, but also influence the patency and remodelling of the environment (extracellular matrix, smooth muscle, blood vessels) given the close anatomic disposition of lung mast cells with airway, smooth muscle and vasculature (1, 6, 7, 10, 24-30).

Although the interaction of IgE with its specific allergen is important, the relationship between IgE and its receptors on cells is critical in allergic asthma. The hallmark of an allergic response is immediate hypersensitivity where IgE binds two cell surface receptors (FcERI and FcERII/CD23), although immediate hypersensitivity is mediated mainly via FcERI (also known as the high affinity IgE receptor) (1-3, 13, 14, 19-24, 31-43). Crosslinking of IgE-bound FcERI, especially on mast cells, leads to the early phase of the allergic response, involving mast cell degranulation and the synthesis of lipid mediators, cytokines and chemokines (1-3, 13, 14, 19-24, 31-43). These proteins will then initiate the late phase which peaks a few hours later and involves the recruitment and activation of other inflammatory cells at sites sensitive to allergen.

Allergens also activate IgE-sensitised antigen-presenting cells, like dendritic cells, which in turn promote IgE production by B-cells to replenish the IgE consumed in the allergic reaction, thereby maintaining mast cell and antigen presenting cell sensitisation (1-3, 6, 7, 11-14, 19-24, 27, 31-51). FcERI is highly expressed on tissue mast cells, quite likely as a result of IgE-mediated upregulated FcERI expression (1-3, 6, 7, 11-14, 19-24, 27, 31-51). FcERI is expressed as an abg2 tetramer on mast cells and basophils and as an ag2 trimer on human but not mouse monocytes, eosinophils and smooth muscle cells (13, 14, 20, 23-25, 28, 52-54).

IgE also binds to a low affinity receptor termed FcERII, and identified as CD23 (35-37, 39, 40, 55, 56). A C-type lectin, CD23 is constitutively expressed on B-cells, airway smooth muscle cells, dendritic cells, lung alveolar macrophages, neutrophils and eosinophils (35-37, 39, 40, 55, 56). IgE acts as a positive regulator of CD23 cell surface expression, just as is the case for FcERI and so does IL-4 (35-37, 39, 40, 55, 56). CD23 activation results in IL-1b, IL-6 and TNF-alpha production by macrophages and dendritic cells and IgE binding on B-cells and dendritic cells enhances the uptake, processing and presentation of allergen epitopes to allergen-specific T-cells and B-cells (35-37, 39, 40, 55, 56). Degranulation and production of autacoids is dependent on immediate signaling of FcERI, however, late signaling events require transcriptional activation and maintenance of gene expression of autacoids and enzymes involved in their biosynthesis (1, 11, 25-27, 57).

NF-kB is a critical regulator of gene transcription in inflammation and is responsive to FcERI signaling in mast cells and dendritic cells (11, 17, 44, 46, 57-59). NF-kB consists of a number of homodimeric and heterodimeric proteins that belong to the Rel family and collectively transactivate proinflammatory genes in a wide variety of immune cells including mast cells, basophils, dendritic cells and lymphocytes (60-64). NF-kB signaling is controlled by a complex series of kinase networks at multiple levels in response to extracellular receptor and non-receptor signals (60-64). Pharmacologic inhibitors of NF-kB and upstream kinases are currently at various stages of development for inflammatory disease including allergy (17, 44, 46, 47, 57, 58, 60-68). Many of the proinflammatory cytokines and enzymes involved in autacoid synthesis expressed in activated mast cells, basophils and dendritic cells are regulated by NF-kB (17, 44, 46, 47, 57, 58, 60-68) and are downregulated in response to NF-kB inhibitors.

Given the role of the IgE molecule in the asthmatic inflammatory response, reducing its levels or inhibiting its actions on mast cells, basophils and dendritic cells could have a major impact on asthma prevention and therapy of active disease. A humanised monoclonal antibody directed against IgE is now in clinical use (Omalizumab; Xolair) and an anti-CD23 antibody (IDEC-152) is also in development after exhibiting a good safety profile (3, 5, 9, 19, 22, 34, 36-38, 42, 43, 55). These therapies, attractive as they are, do not completely eliminate IgE levels. “Escaped” IgE can therefore still activate acute processes in mast cells, basophils and dendritic cells. In alternate approaches, antisense DNA technology has been used to downregulate FcERI and NF-kB (44, 69-72) or other molecules involved in allergic asthma (Respirable AntiSense OligoNucleotides; “RASONs”; 73-78), but successful application of these methodologies has been impeded by the short half-life of ODNs and the need for an effective concentration of molecules to accumulate in lung target tissue.

### 3. SUMMARY OF THE INVENTION

The present invention relates to methods and compositions for inhibiting inflammatory processes (such as asthma) in the lungs, wherein oligonucleotides (“ONs”) targeting IgE receptors and NFkappaB are administered via an aerosolized microsphere formulation. It is based, at least in part, on the discoveries that (i) when this mixture of ONs is administered to mast cell and dendritic cell lines in vitro, the production of proinflammatory cytokines is considerably attenuated, and (ii) microspheres were found to reach a large population of lung cells following intratracheal instillation.

### 4. BRIEF DESCRIPTION OF THE FIGURES

FIG. 1. Fluorescence activated cell sorting (“FACS”) measurement of FcERI downregulation on the surface of MC9 mastocytoma cells, following incubation of the cells with DNP-specific mouse IgE and crosslinking with DNP-BSA (at 8 hours).

FIG. 2. Incubation of bone marrow derived dendritic cells from BALB/c and C3H/HeJ mouse strains in vitro with NFkB decoy oligonucleotide downregulates cell surface FcERI (10 micromolar final oligo concentration in culture).

FIG. 3A-B. (A) AS-FcERI downregulation of FcERI on MC9 and P815 cell lines in vitro. Cells were treated with 2 μM (final) mix of (1:1) antisense FcERI plus antisense CD23 overnight. Parallel cultures were then treated with DNP-specific IgE followed by DNP-BSA. Five hours later, cells were stained with specific anti-FcERI or CD23 fluorescently tagged antibodies. FACS analysis was then used to determine mean fluorescence intensity (geometric mean) representing mean levels of receptor. Error bars represent the SEM of triplicate parallel cultures, (B)Phospho-SyK content in AS-FcERI treated cell lines in vitro. Cells were treated with 2 μM (final) mix of (1:1) antisense FcERI plus antisense CD23 overnight. Parallel cultures were then treated with DNP-specific IgE followed by DNP-BSA. Five hours later, cells lysates (clarified) were scanned for phosph-Syk using Luminex technology. Data shown as fold-increase in phospho-Syk content over untreated cell p-Syk content (error bars are SEM of triplicate wells).

FIG. 4A-B. (A) Fluorescence imaging of lungs excised from mice administered fluorescent microspheres intratracheally, 24 hours post-administration. The lungs and surrounding tissue at the left are from mice administered vehicle control. The lungs and surrounding tissue on the right are from mice administered the fluorescent microspheres. Imaging was performed using an IVIS Lumina system. (B) Fluorescent microsphere accumulation in bronchoaviolar lavage cells following intratracheal instillation. Cells were isolated from the bronchoaviolar lavage fluid (“BALF”) of the mice whose lungs are shown in FIG. 4A and stained for CD11b or FcERI, FACS was performed on the cells isolated 30 minutes or 24 hours after microsphere instillation inside the trachea. Percent gated cells refers to lice cells in the total BALF collected. FL refers to microsphere fluorescence and Q2 refers to the upper right quadrant of the FACS plot.

FIG. 5. Histamine concentration in BAL fluid from mice with or without OVA-induced asthma treated with either PBS alone (“no particles”), empty microspheres or microspheres loaded with FcER1a antisense oligonucleotides.

FIG. 6A-C. Cytokine levels in broncheolar lavage fluid (“BALF”) of mice, either with or without OVA_induced asthma, treated with either PBS (“nothing”), unloaded microspheres (“empty”), or FcER1-loaded microspheres (“fcer1”). (A) G-CSF, GM-CSF, IFNg, IL-1b, IL-2, IL-4, IL-5, and IL-6; (B)IL-9, IL-10, IL-12(p70), IL-13, IL-15, IL-17, IP10, and KC; (C) MCP-1, MIP-1a, RANTES, and TNFa.

### 5. DETAILED DESCRIPTION OF THE INVENTION

For purposes of clarity, and not by way of limitation, the detailed description of the invention is divided into the following subsections:

(i) therapeutic ONs;

(ii) microsphere formulations; and

(iii) methods of treatment.

### 5.1 Therapeutic ONs

ONs that may be used according to the invention may target the high-affinity IgE receptor FcERI, the low-affinity IgE receptor CD23, or the p65 subunit of NfkappaB. For human therapeutic applications, preferably the human mRNA sequence is the basis for the target, but the invention also provides for use of non-human sequences that may be used either in animal model systems for confirmation of therapeutic effects and for dosage adjustment, for example, but not limited to, the murine sequence or a related rodent sequence, or for the treatment of non-human animals suffering from airway inflammation.

FcERI occurs on cells as a tetramer composed of an alpha, beta, and two disulfide-linked gamma chains. Genes encoding any of these subunits may be targets according to the invention. As one non-limiting example, the target may be the human alpha subunit for example, and not by way of limitation, having at least a portion of the sequence as set forth in SEQ ID NO: 5. As another non-limiting example, the target may be the murine alpha subunit for example, and not by way of limitation, having at least a portion of a sequence as set forth in SEQ ID NO: 6. As another non-limiting example, the target may be the human beta subunit for example, and not by way of limitation, having at least a portion of a sequence as set forth in SEQ ID NO: 7. As another non-limiting example, the target may be the murine beta subunit for example, and not by way of limitation, having at least a portion of a sequence as set forth in SEQ ID NO: 8 or a sequence which is at least 95 percent homologous thereto. As another non-limiting example, the target may be the human gamma subunit for example, and not by way of limitation, having at least a portion of a sequence as set forth in SEQ ID NO: 9. As another non-limiting example, the target may be the murine gamma subunit for example, and not by way of limitation, having a sequence as set forth in SEQ ID NO: 10.

In further non-limiting embodiments of the invention, an ON may target CD23. As one non-limiting example, the target may be human CD23 for example, and not by way of limitation, having at least a portion of a sequence as set forth in SEQ ID NO 11: As another non-limiting example, the target may be murine CD23 for example, and not by way of limitation, having at least a portion of a sequence as set forth in SEQ ID NO: 12.

In further non-limiting embodiments of the invention, an ON may target NFkappaB. As one non-limiting example, the target may be human NFkappaB for example, and not by way of limitation, having at least a portion of a sequence as set forth in SEQ ID NO: 13. As another non-limiting example, the target may be murine NfkappaB for example, and not by way of limitation, having at least a portion of a sequence as set forth in SEQ ID NO: 14. In further non-limiting embodiments of the invention, an ON may target a binding sequence of the NF-KappaB transcription factor where the ON is a decoy for the CONSENSUS BINDING SEQUENCE OF NF-kappaB and its homologues (i.e. RelB and all other transcription factors binding to the NF-kappaB consensus sequence (SEQ. ID 5′-CTGGGGACTTTCCAT-3′ (SEQ ID NO:1)); for example but not by way of limitation the ON may comprise (5′-CTGGGGACTTTCCAT-3′ (SEQ ID NO:1)), for example in a double stranded duplex where a first strand comprises an ON 1=5′-CTGGGGACTTTCCAT-3′ (SEQ ID NO:1) and a second strand comprises ON2=5′ ATGGAAAGTCCCCAG 3′ (SEQ ID NO:2).

An ON according to the invention may be entirely or predominantly comprised of deoxyribonucleotides (an “ODN”) or entirely or predominantly comprised of ribonucleotides (an “ORN”), and may comprise one or more modified nucleotide, for example, nucleotides joined via a phosphorothioate bond. An ON may be an antisense ON, a catalytic ON, or an RNAi or siRNA ON, as are known in the art. An ON may be single stranded or double stranded or have single stranded as well as double stranded portions. In preferred non-limiting embodiments, the ON is an ODN.

An “antisense ON” refers to an ON that is complementary to at least a portion of a primary transcript or mRNA and that inhibits the expression of a target gene (see U.S. Pat. No. 5,107,065). The complementarity of an antisense ON may be with any part of the specific gene transcript, i.e., at the 5′ non-coding sequence, 3′ non-coding sequence, introns, or the coding sequence. An antisense ON may be between about 10 and 100 nucleotides, or between about 10 and 50 nucleotides, or between about 15 and 100 nucleotides, or between about 15 and 50 nucleotides, or between about 10 and 35 nucleotides, or between about 15 and 35 nucleotides.

A catalytic ON is an ON that has enzyme catalytic activity and comprises a portion homologous to its target gene. For example, but not by way of limitation, a catalytic RNA may be referred to as a “ribozyme” and may catalyze hydrolysis and/or aminotransferase activity.

siRNA typically comprises a polynucleotide sequence identical or homologous (e.g., at least 90 percent or at least 95 percent or at least 98 percent homologous) to a portion of a target gene linked directly, or indirectly, to a polynucleotide sequence complementary to the sequence of the target gene (or fragment thereof) (see, for example, Hunter, 1999, Curr. Biol. 9:R440-442; Hamilton el al., 1999, Science 286:950-952; Ding, 2000, Curr. Opin. Biotechnol. 11:152-156). In a nonlimiting example, a RNAi or siRNA molecule may be between about 6 and 50 nucleotides. In particular nonlimiting examples, the RNAi or siRNA molecule is between about 10 and 35 nucleotides, or between about 10 and 25, or between about 15 and 25 nucleotides. An siRNA may optionally comprise a polynucleotide linker sequence of sufficient length to allow for the two polynucleotide sequences to fold over and hybridize to each other; however, a linker sequence is not necessary. The linker sequence is typically designed to separate the antisense and sense strands of siRNA sufficiently as to limit the effects of steric hindrance and allow for the formation of dsRNA molecules and preferably does not hybridize with sequences within the hybridizing portions of the siRNA molecule.

Ribozymes, antisense polynucleotides, and siRNA molecules may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand {or strands); the promoters may be known inducible promoters such as baculovirus. Inhibition may be targeted by specific transcription in an organ, tissue, or cell type. The RNA strands may or may not be polyadenylated; the RNA strands may or may not be capable of being translated into a polypeptide by a cell's translational apparatus.

The ON may be chemically or enzymatically synthesized by manual or automated reactions. The ON may be synthesized by a cellular nucleic acid polymerase or a bacteriophage polymerase (e.g, T3, T7, SP6). If synthesized chemically or by in vitro enzymatic synthesis, the ON may be purified prior to use. For example, an ON can be purified from a mixture by extraction with a solvent or resin, precipitation, electrophoresis, chromatography, or a combination thereof. The ON may be dried for storage or dissolved in an aqueous solution. The solution may contain buffers or salts to promote annealing, and/or stabilization of the duplex strands.

Homology may be determined using standard techniques, for example using software known in the art such as BLAST or PASTA.

Specific, non-limiting examples of ON that may be used according to the invention include: homologous FcERI-alpha (human/mouse from 3′ end of sequence): gtg tee aca gca aac aga ate (SEQ ID NO:3); homologous (mouse/human) CD23 (also from 3′ end): gca gaa ggc gte gtt cca {SEQ ID NO:15); mouse FcERI-alpha: cca gtg acc ate gcc ect g (SEQ ID NO:16); mFcERIa control: cag ggg cga tgg tea ctg g (SEQ ID NO:17); hFcERlalpha-1: aca gta gag tag ggg att cca tgg cag gag cca tct tct tea tgg act cc (SEQ ID NO:18); hFcERlalpha-2: tte aag gag ace tta ggt ttc tga ggg act get aac acg cca tet gga ge (SEQ ID NO:19); and hFcERII (CD23): tet ctg aat att gae ctt cct cca tgg egg tee tgc ttg gat tet ccc ga (SEQ ID NO:20).

Activity of an ON according to the invention may be determined by an in vitro assay, for example in a cell-free system or cell culture, to test the ability of the ON to inhibit the expression of its target protein. As specific, non-limiting examples, validation of inhibitory activity may be performed using rodent and human mast cell and basophil cell lines such as LAD2, P815, Ku812 and MC9 by FACS ascertainment of surface levels of target and by steady state quantitative RT-PCR and/or Western blot analysis. Dendritic cells lines may also be used in the testing. As one potential criterion, an ON may be selected for use or further study based on its demonstrating, in cell culture, relatively less , off-target knockdown activity using genome-wide expression array technology on cell line transcripts (88-91). In addition or alternatively, the activity of the ON may be assessed by determining its affect on autacoid and cytokine production in culture supernatants, for example using ELISA/Luminex 24 hours following FcERI crosslinking using IgE:DNP-HSA exposure, or by ascertaining nuclear p65 NF-kB levels by standard mobility shift assay (49, 51, 68, 69, 79, 92-97).

Additionally, activity of an ON may be confirmed using an in vivo assay. For in vivo testing an ON may preferably be comprised in a microsphere, as discussed below. As a specific, non-limiting example, ON, preferably comprised in microspheres, may be administered to Balb/c mice by aerosol into the respiratory tract using a Bennet nebuliser. The mice may be placed inside a Plexiglas chamber and may be administered 10-200 micrograms of dry ON-containing powder per mouse, where the average diameter of each ON-containing particle is preferably about 1-2 microns and the flow rate may be from 10-20 liters/min for 20-60 minutes. Comparisons may be made to mice exposed to formulated “scrambled” oligonucleotide sequences and, where microspheres are used, to “empty” microspheres. At various times after treatment, mice in the treatment and non-treatment groups may be euthanized and the effectiveness of ON at inhibiting expression and/or functionality of target may be assessed, for example by measuring the levels of FcERI alpha, beta and gamma chain, CD23 and p65 NF-kB steady state mRNA in bronchiolar lavage fluid cells and/or by FACS measurement of cell surface FcERI a chain and CD23 in parallel cells. Efficacy may also be assessed in bioassays in vitro, where bronchiolar lavage fluid cells isolated from treated mice may be exposed to FcERI (FcERI crosslinking by IgE-dinitrophenyl-HSA exposure) and NF-kB activation (exposure to LPS) followed by cytokine and autacoid measurement in culture supernatant along with ascertainment of the degree of FcERI-dependent intracellular signaling (phospho-Syk, phospho-Lyn by Western blot) (12, 49-51, 68, 69, 79, 92-106).

Effectiveness of an ON may also be assessed in a model for asthma, for example but not limited to the ovalbumin-induced and the house dust mite allergen-triggered models of acute and chronic allergic asthma (reviewed in 107, 108). Specifically, airway function may be measured using the flow interrupter method on the basis of the response of total respiratory system resistance to saline and increasing doses of intravenous methacholine as well as whole body plethysmography to calculate enhanced pause response to methacholine (12, 50, 98-106). In addition to these physiologic measurements of airway function, lung histology may be assessed and morphometry may be performed to determine extracellular matrix (and collagen) deposition and remodeling, airway epithelium mucus content, smooth muscle cell proliferation, density and morphometry of goblet cell hyperplasia.

### 5.2 Microsphere Formulations

The present invention provides for microspheres that carry one or more ON, as described above, and for formulations comprising said microspheres.

In certain embodiments, microspheres carry one or more species of ON that targets the high-affinity IgE receptor FcERI.

In certain embodiments, microspheres carry one or more species of ON that targets the low-affinity IgE receptor CD23.

In certain embodiments, microspheres carry one or more species of ON that targets the p65 subunit of NfkappaB.

According to certain embodiments of the invention, a plurality of (two or more) different ONs as described above are comprised in a microsphere formulation. In such embodiments, an individual microsphere may carry only one species of ON, and a therapeutic formulation may comprise a mixture of microspheres bearing different ONs, or, alternatively, an individual microsphere may itself carry a mixture of ONs, or, alternatively, a combination of microspheres some carrying one species of ON and others carrying more than one species of ON may be used.

A microsphere is a particle having a diameter of between about 10 nanometers and 10 micrometers or between about 100 nanometers and 1 micrometer or between about 200 nm-2 micrometers or between about 200-400 nanometers.

ON-carrying microspheres may be prepared by methods known in the art. In particular, non-limiting embodiments, the microspheres may be prepared using a method as set forth in Kovacs et al., 2009, J. Biomaterials Science 20:1307-1320 or Zheng et al., 2006, J. Biomater. Sci. Polymer Edn. 17(12):1389-1403, both incorporated by reference in their entireties herein.

In particular, non-limiting embodiments of the invention, polycation-coated poly(D,L-lactide-co-glycolide) (“PLGA”) microspheres may be used as carriers of ONs into the lungs. The PLGA microspheres preferably have an average hydrodynamic size between 300-500 nm, such that they may be rapidly internalized by phagocytic cells and are sufficiently small to pass through conducting airways to reach the deep lung, yet with density high enough to prevent them from being exhaled (for example, a density of between about 0.5-1 g/cm³. In particular non-limiting embodiments, the polycation may be a peptide. Said cationic peptide may be of a length between about 8 and 25 amino acids and may preferably comprise between about 10 and 20 cationic amino acid residues such as histidine, ornithine or ariginine to facilitate ODN uptake and to promote ODN escape from endosomes or phagosomes upon internalization (80). In a specific, non-limiting embodiment, the cationic peptide comprises Orn-Orn-Orn-Orn-Orn-Orn-Orn-Orn-Orn-Orn-His-His-His-His-His-His (SEQ ID NO:4).

In one specific, non-limiting embodiment, the microsphere may be a PLGA-Ni particle prepared using a double emulsion (w/o/w) solvent evaporation method. 90 mg PLGA and 0.6 mg 1,2-dioleoyl-m-glycero-3-((N(5-amino-1-carboxypentyl)-imino-diacetic acid]-succinyl) nickel salt)) (“DOGS-NTA-Ni”) may be dissolved in 3 ml methylenechloride. To this organic phase 200 μL double distilled water may be added and the mixture sonicated (20 W) for 2 minutes. This primary emulsion may be added to 20 ml double distilled water and the resulting mixture may be allowed to evaporate for four fours to remove the methylenechloride. Particles may be washed twice in double distilled water and recovered by centrifugation. The resulting solids may be confirmed using Fourier transform infrared spectroscopy to contain DOGS-NYA-Ni. ON (preferably ODN)loading may be accomplished by first coating the particles (30 μL from 4.5 mg/ml stock) with O10H6 (ornithine (10)-histidine (6); 60 μg), SEQ ID NO:4 followed by addition of one or more type of ON (0.06 nmoles) in 300 μl double distilled water. Equilibration of each component may be achieved using gentle shaking for 30 minutes at room temperature.

In another specific, non-limiting embodiment of the invention, PLGA/O10H6 microspheres may be prepared using a double emulsion (w/o/w) solvent evaporation technique (Jain, 2000, Biomaterials 21:2475). 5 μg O10H6 may be added to 200 μL of an aqueous solution containing 40 μg of one or more type of ON (preferably an ODN) and gently shaken for 0.5 hours at room temperature. This aqueous solution may be incorporated into a methylene chloride solution of PLGA (3% w/v) by sonication. A Fisher model 100 sonic microprobe may be used to introduce 24 W of energy (over 2 minutes) to form this water-in-oil emulsion. The primary emulsion may be added drop-wise into 20 ml of an aqueous solution of PVA (0.5% w/v) to form a water-in-oil-in-water emulsion. The resulting double emulsion may be stirred for about 4 hours in a chemical fume hood to allow the methylene chloride to evaporate. Particles may be recovered by ultracentrifugation and washed twice with double distilled water to remove excess PVA and un-trapped ON.

ON-carrying microspheres may optionally be freeze-dried, stored, and reconstituted in distilled water. Addition of sucrose (2% w/v) prior to freeze-drying may improve recovery.

The formulation comprising ON-carrying microspheres may be a suspension, an emulsion, or a dry powder.

Formulations to be used therapeutically may further comprise a suitable pharmaceutical carrier , optionally a preservative, and optionally one or more additional active agent, for example, but not limited to, a bronchodilator such as theophylline, albuterol, salmeterol, formoterol, a leukotriene modulator such as Montelukast, Zafirlukast, or Zileuton, and/or an anti-inflammatory agent such as fluticasone, budesonide, mometasone, beclomethasone, or ciclesonide and/or a mast cell stabilizer such as cromolyn or nedocromil, or an antibiotic, in therapeutically beneficial concentrations. The formulation may optionally comprise a propellant and/or additional excipients as appropriate.

### 5.3 Methods of Treatment

In certain non-limiting embodiments, the present invention provides for a method of treating an inflammatory lung disorder comprising administering, to a subject in need of such treatment, an effective amount of microspheres carrying a mixture of ONs comprising at least one ON that inhibits expression of an IgE receptor and at least one ON that inhibits expression of NFkappaB. In specific, non-limiting embodiments of the invention, the following combinations of ON may be used:
an ON targeting FcERI alpha, beta or gamma chain and an ON targeting NFkappaB p65, optionally further comprising an ON targeting CD23.

In certain specific non-limiting embodiments, the present invention provides for a method for reducing an inflammatory response in the lungs, for example in the context of treating asthma, comprising administering, to a subject in need of such treatment, an effective amount of microspheres carrying an ON that targets FcERI alpha. For example, but not by way of limitiation, reduction in the inflammatory response may be measured as a decrease in a cytokine selected from the group consisting of GM-CSF, IFNg, IL-9, IL-15, IL-17, KC, and TNFa.

The subject may be a human subject or a non-human subject such as a non-human primate, a rodent such as a mouse or rat, a dog, a cat, a horse, etc.

Disorders which may be treated according to the invention include but are not limited to asthma (e.g. bronchial asthma) and other IgE-related inflammatory airway disorders.

A formulation comprising an effective amount of ON-bearing microspheres as set forth above may be administered via the airway of a subject in need of such treatment. Administration to the lungs may be performed using devices known in the art, including but not limited to a nebulizer, a metered dose inhaler, or a dry powder inhaler. As an alternative to administration via the mouth, the formulation may be administered through the nose. In a specific, non-limiting embodiment of the invention, the flow rate of delivery of microspheres into the lungs is between about 10-20 L/minute for between about 20-60.

An effective amount of ON means an amount that results is an amelioration of pulmonary performance, for example, but not limited to, a subjective reduction of respiratory distress, a reduced cough, an increase in the forced expiratory volume, and/or an increase in the forced expiratory volume over 1 second/ forced vital capacity ratio, where the increase may be, for example and not by way of limitation, by at least 5 percent, or by at least 10 percent, or by at least 15 percent or by at least 20 percent or by at least 25 percent.

In specific, non-limiting embodiments of the invention, the amount of ON administered per kilogram of a subject may be between about 1-100 or between about 10-50 or between about 1-10.

In non-limiting embodiments of the invention, ON-bearing microspheres may be administered either as needed, or once a day, or at least once a day, or twice a day, or three times a day, or four times a day, or once every other day, or twice a week, or once a week, or once every two weeks, or once a month. Treatment can be on a one-time basis, using a regimen as described in the previous sentence for a limited period of time, continuously, or using a regimen interrupted by a period of non-treatment.

### 6. EXAMPLE: IN VITRO ON STUDIES

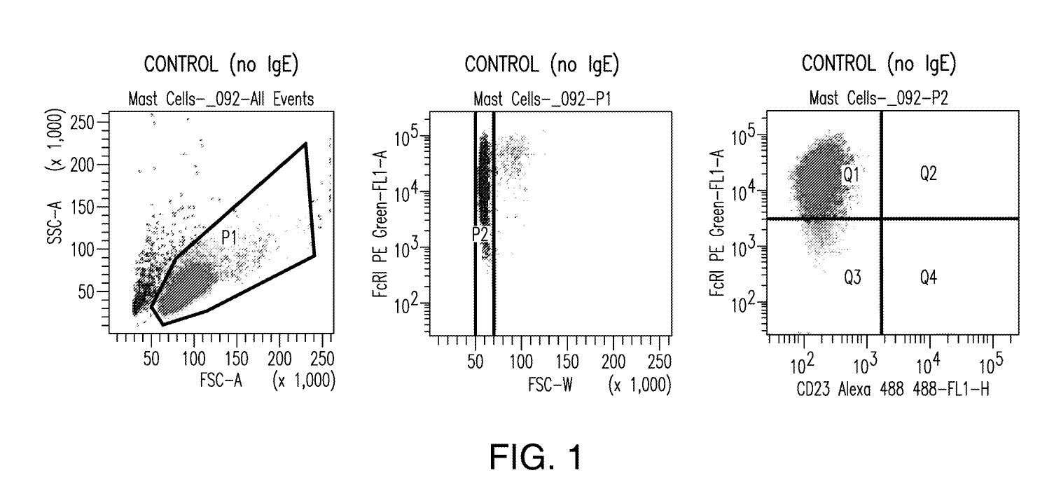

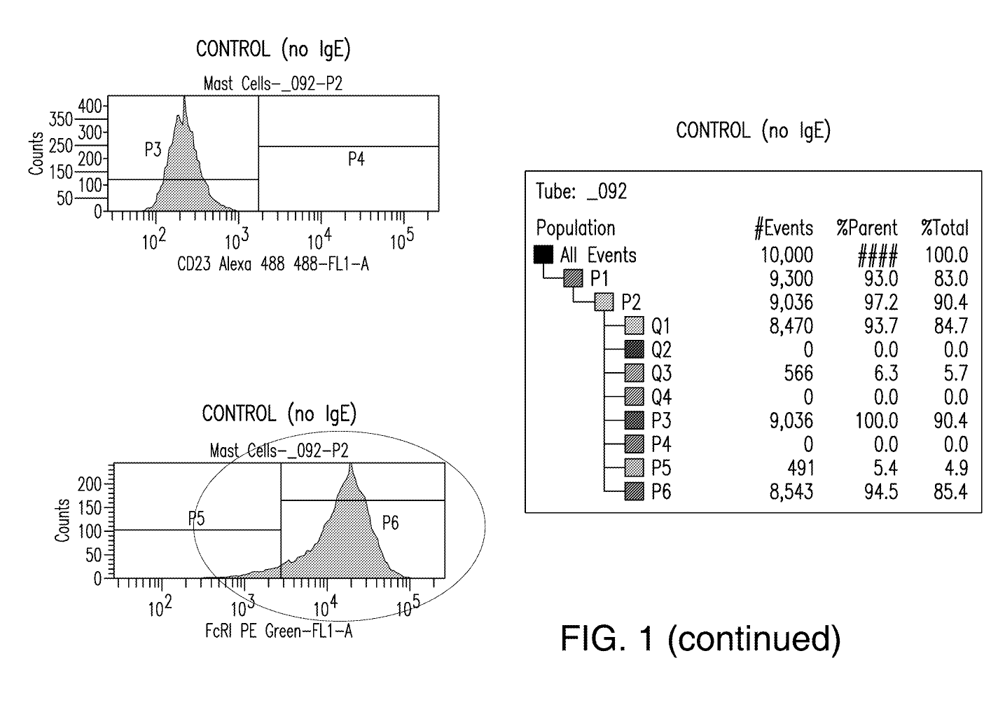

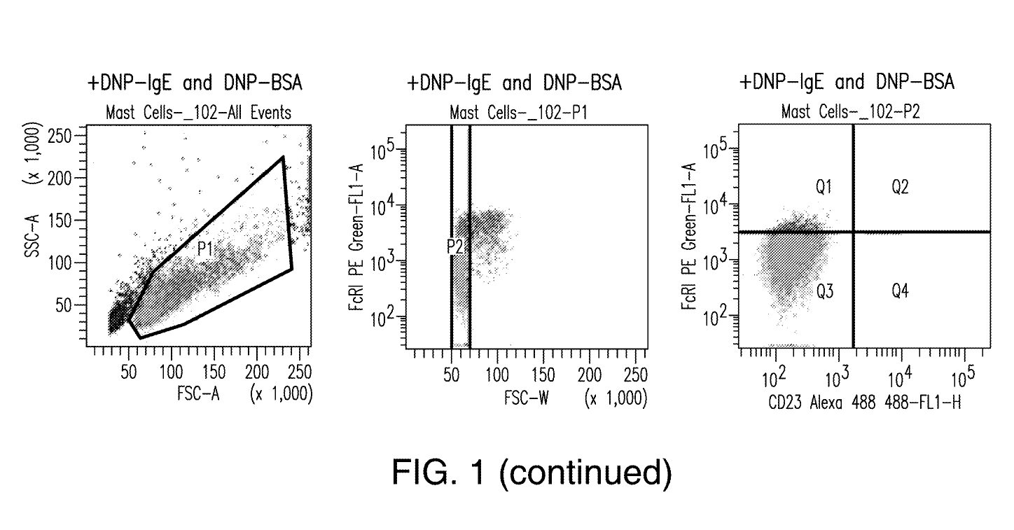

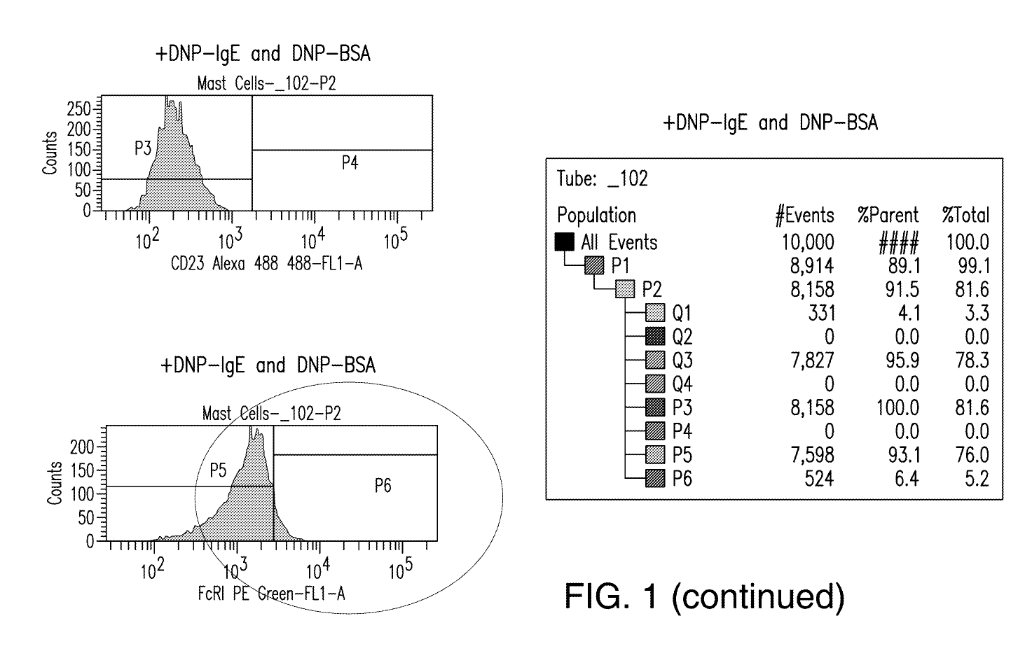

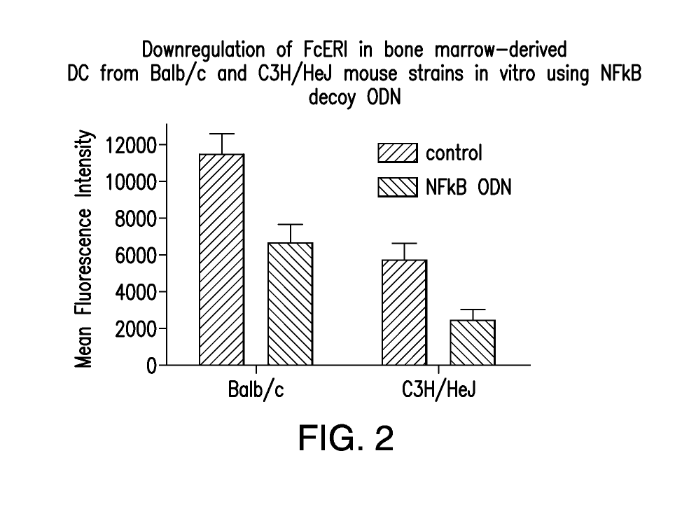

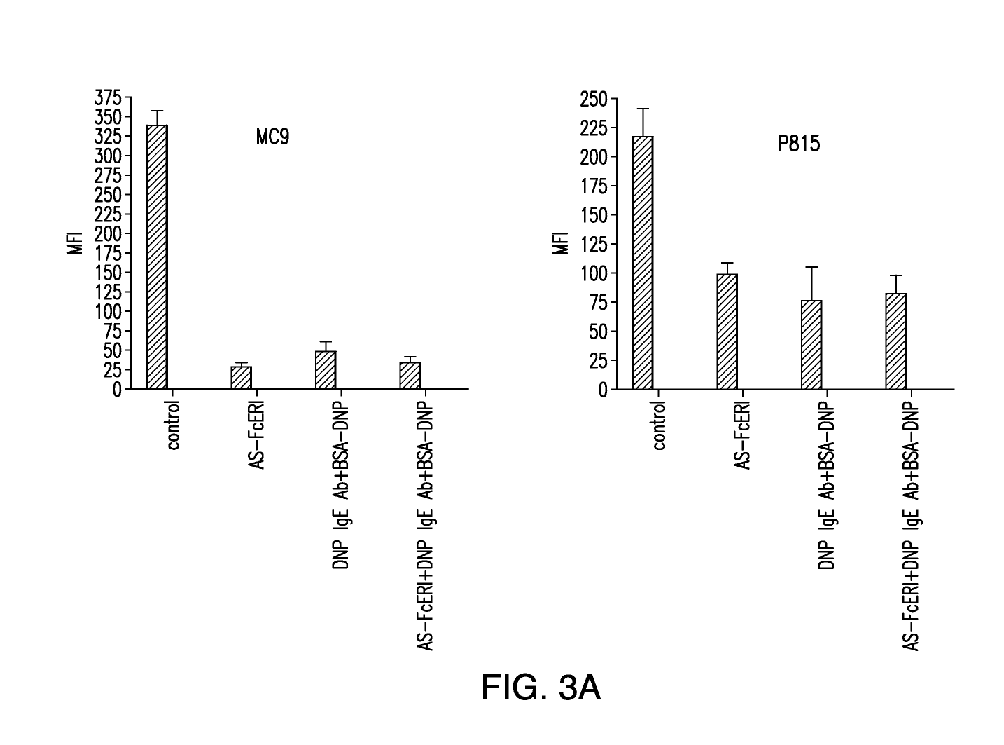

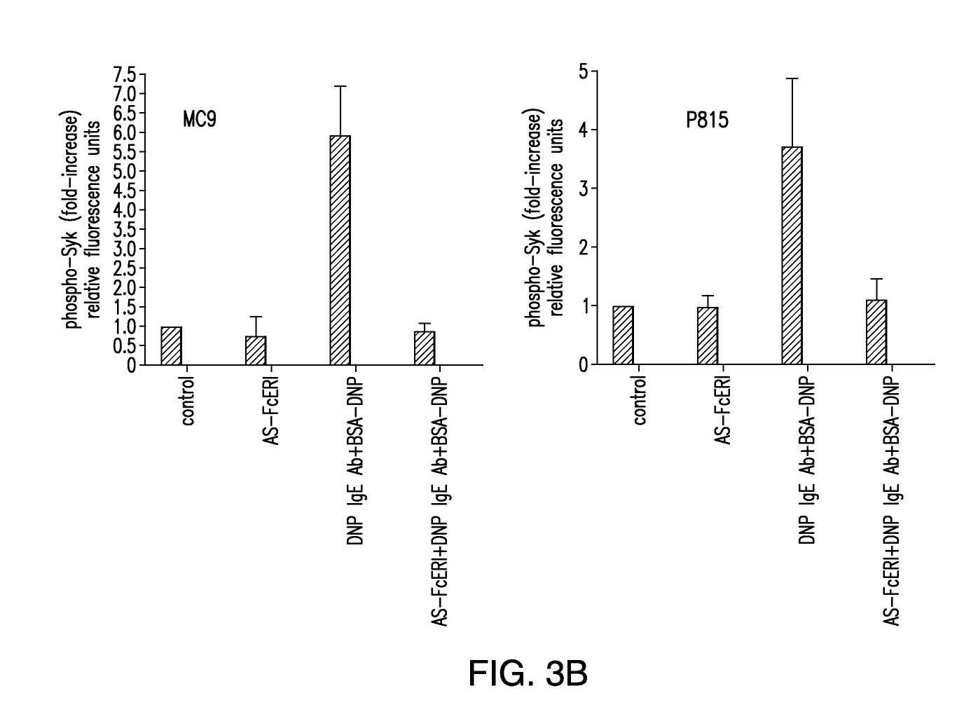

FIG. 1 shows FACS measurement of FcERI downregulation on the surface of MC9 mastocytoma cells, following incubation of the cells with DNP-specific mouse IgE and crosslinking with DNP-BSA (51, 69, 79). FIG. 2 shows that incubation of bone marrow derived dendritic cells from two mouse strains in vitro with NFkB decoy oligonucleotide having SEQ ID NO: 1 downregulates cell surface FcERI (10 micromolar final oligo concentration in culture). FIG. 3 shows that incubation of two relevant cell lines (MC9 mastocytoma and P815 basophil-like cells) in vitro with a combination of antisense FcERI (SEQ ID NO:2) and antisense CD23 oligonucleotide (SEQ ID NO:3) downregulates cell surface FcERI (FIG. 3A) and CD23 and is associated with suppressed tyrosine phosphorylation of FcERI-dependent Syk even when FcERI is bound by DNP-specific IgE subsequently crosslinked by BSA-DNP (FIG. 3B; by Luminex detection of phospho-specific Syk xMAP bead fluorescence; 2 micromolar final each oligo concentration in culture).

### 7 EXAMPLE: IN VIVO STUDIES

Microspheres made with poly-(d,l-lactide-co-glycolide), or PLGA, SEQ ID NO. 4 loaded with ODN were prepared by a double emulsion solvent evaporation method. 90 mg PLGA was dissolved in 3 ml MeCl₂. To this organic phase 200 μL deionized water was added and sonicated (20 W) for 2 min. This primary emulsion was added to 20 ml of deionized water containing 0.8% polyvinyl alcohol and the resulting mixture was allowed to evaporate for 4 h to remove MeCl₂. Particles were washed twice in deionized water and recovered by centrifugation. Particles coated with SEQID NO. 4 were loaded with ODN.

The resulting microsphere had an average diameter of 280 nm with ρ˜1 g/cm³.

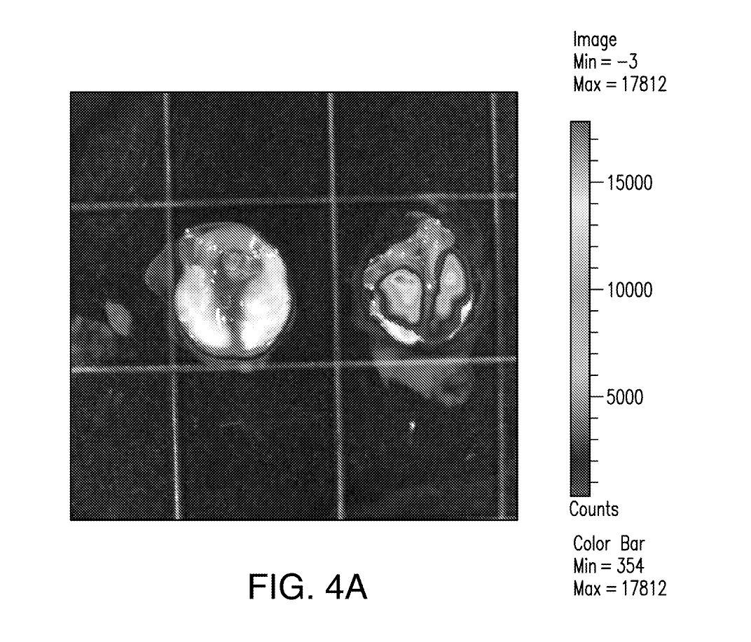

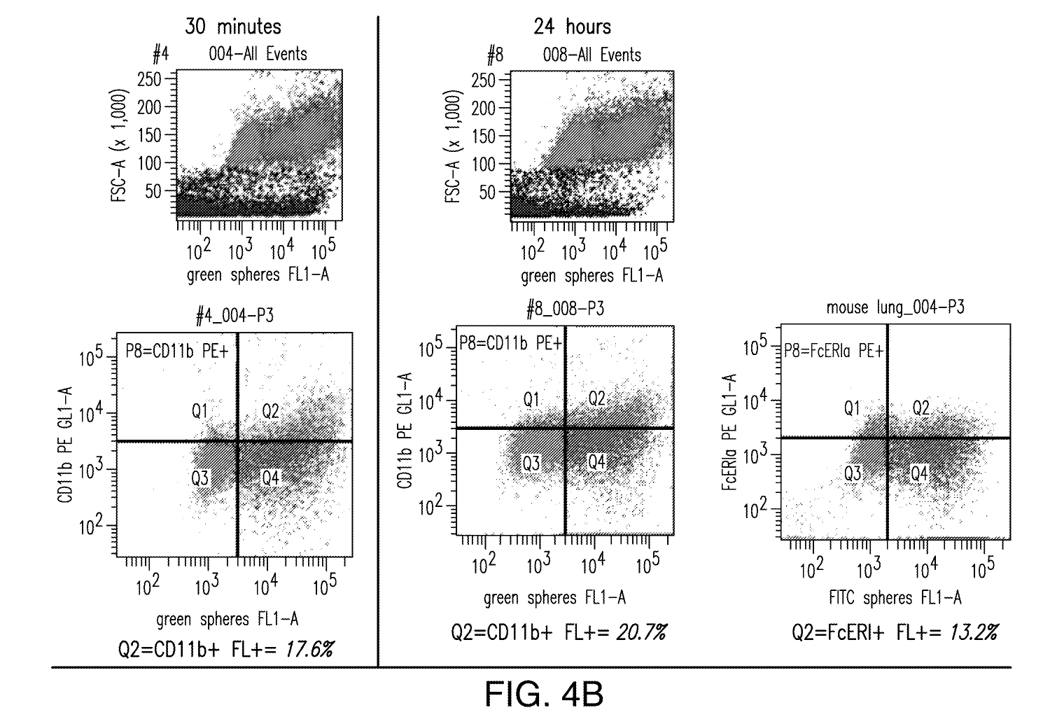

FIG. 4A demonstrates that after intratracheal instillation into female Balb/c mice of 6-12 weeks of age, the microspheres populate almost the entire lung tissue and the accumulation is stable for at least 24 hours. FIG. 4B shows that in the bronchalveolar lavage fluid (“BALF”) of the mice from which the lungs shown in FIG. 4A derive, microspheres are captured by macrophage/monocytes (CD11b+ cells) as well as a superpopulation of FcERI+cells that very likely includes mast cells, basophils and dendritic cells.

Experiments were then performed to evaluate the effect of FcERI ODN-loaded microspheres in a murine model for asthma induced by ovalbumin (“OVA”) [107, 108]. Briefly, six to twelve week old Balb/c mice were sensitized on day 0 by intraperitoneal injection of 50 ug chick ovalbumin (OVA) and 2 mg alum in 200 microliters PBS. On Day 6 mice were pretreated with 30 microliters (about 50 micrograms) of particle suspension (particles were loaded with antisense FcERIa oligonucleotides at a ratio of 18.5 micrograms DNA per mg particles). The mice were placed under deep anaesthesia by inhalation of 3% isoflurane for about 5 minutes. Mice were held by the scruff of the neck in an upright supine position and 15 microliters of the particle suspension was pipetted on to each nares. Inhalation of the particle suspension was verified by audible glottal clicking as the fluid passed into the trachea. On Days 7, 8, 9, and 10 mice were again treated with 50 micrograms of AS-FcERIa particles. Following treatment, the mice were returned to their cages and allowed to recover. Thirty minutes following treatment with particle suspension, mice were challenged intranasally with 50 micrograms ovalbumin. The procedure is identical to intranasal delivery of particle suspension. Mice received 25 microliters of a 1 microgram/microliter solution of ovalbumin in PBS applied to each nares. On day 10, three hours following the final ovalbumin challenge, bronchoalveolar lavage was performed to recover intrapulmonary exudate and leukocytes.

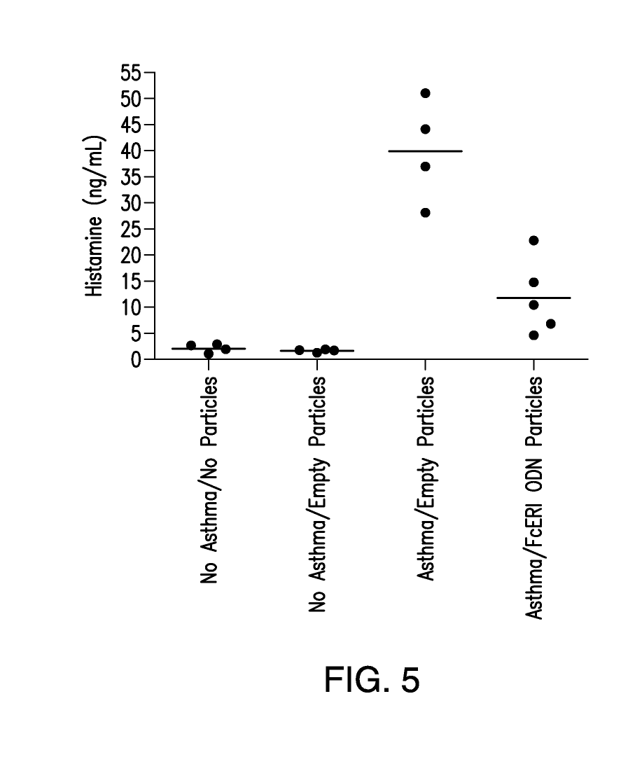

In a first series of studies, the amount of histamine present in BALF was compared in mice either with or without OVA-induced asthma as early as 24 hours following the last OVA challenge, treated or untreated with FcERIa ODN microspheres. As can be seen in FIG. 5, where the negative controls are mice without OVA-induced asthma that were not treated with any particles (they were given phosphate buffered saline, “PBS”) or were treated with empty particles, there was a substantial increase in the histamine level in mice with OVA-induced asthma sham-treated with empty particles, but this increase was reduced by more than half in animals with OVA-induced asthma treated with FcERIa antisense ODN-carrying microparticles.

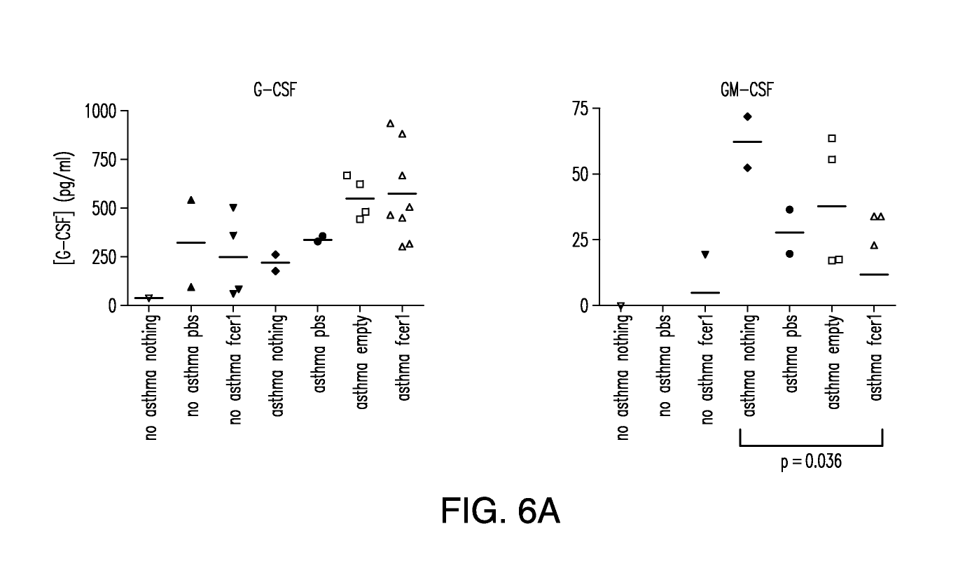

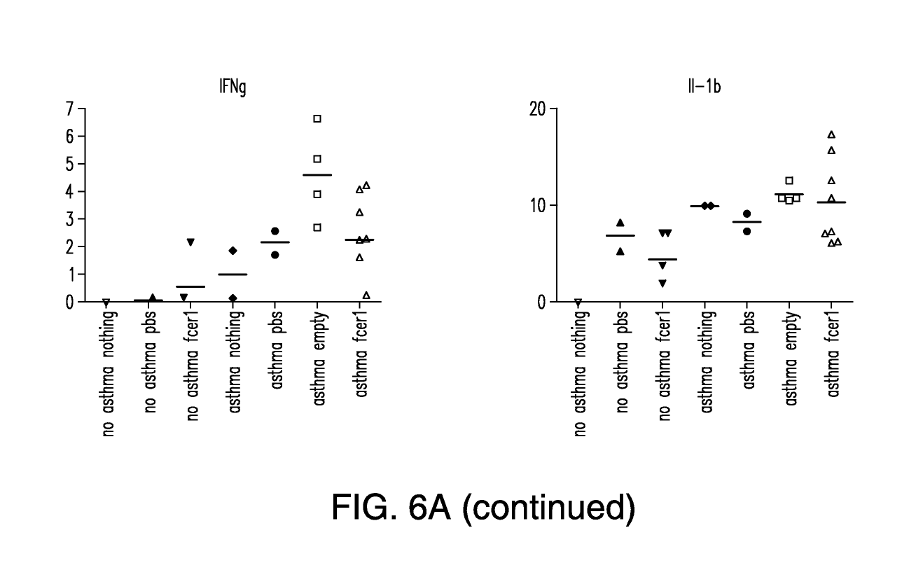

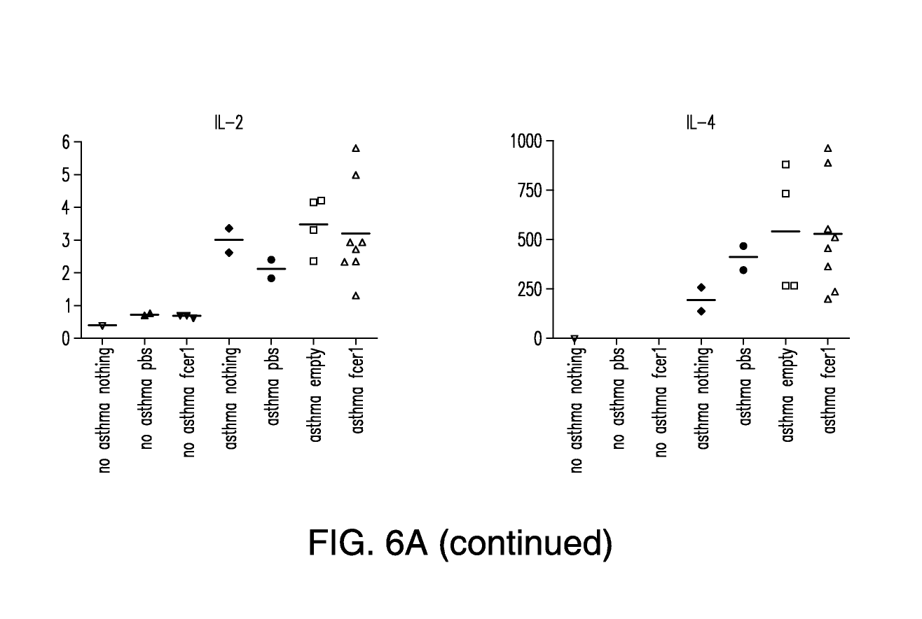

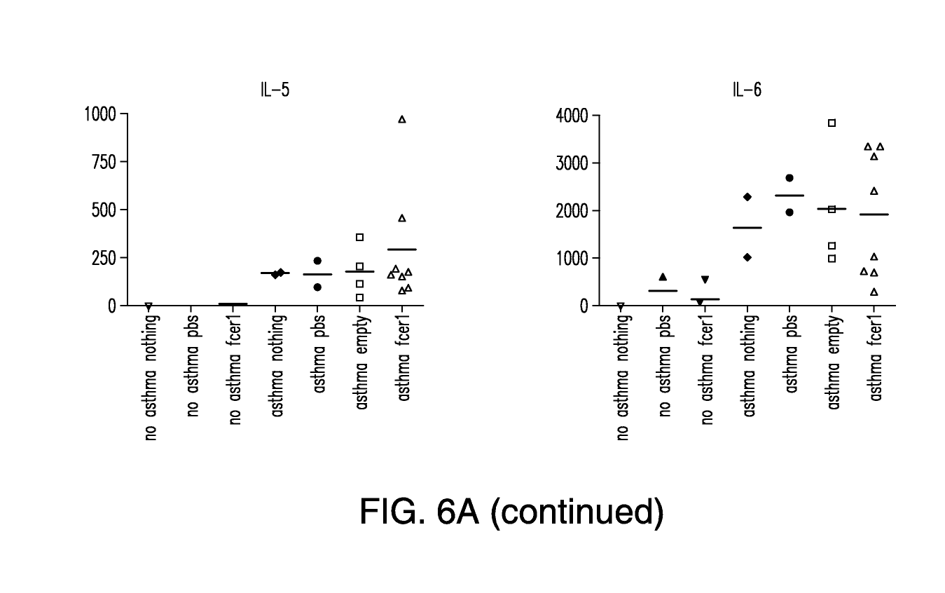

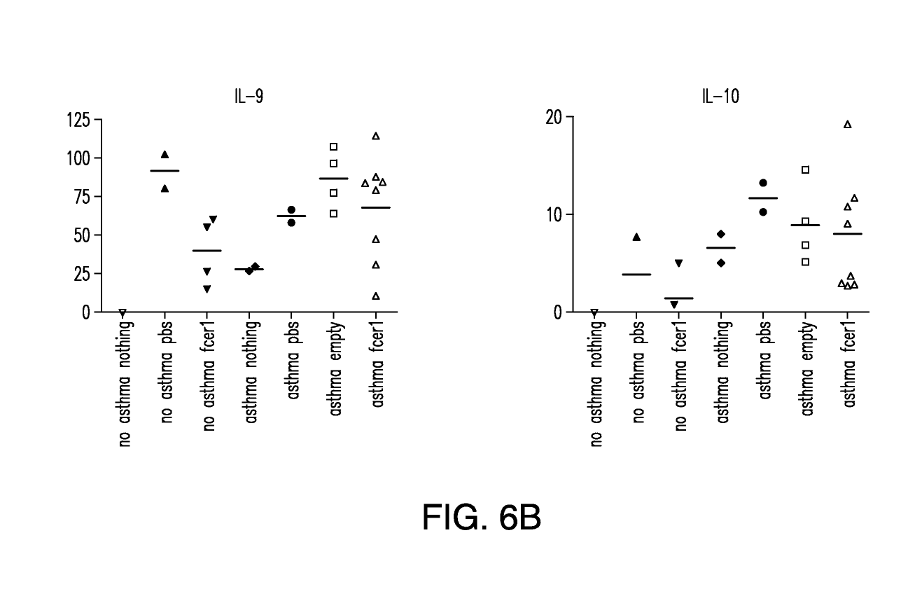

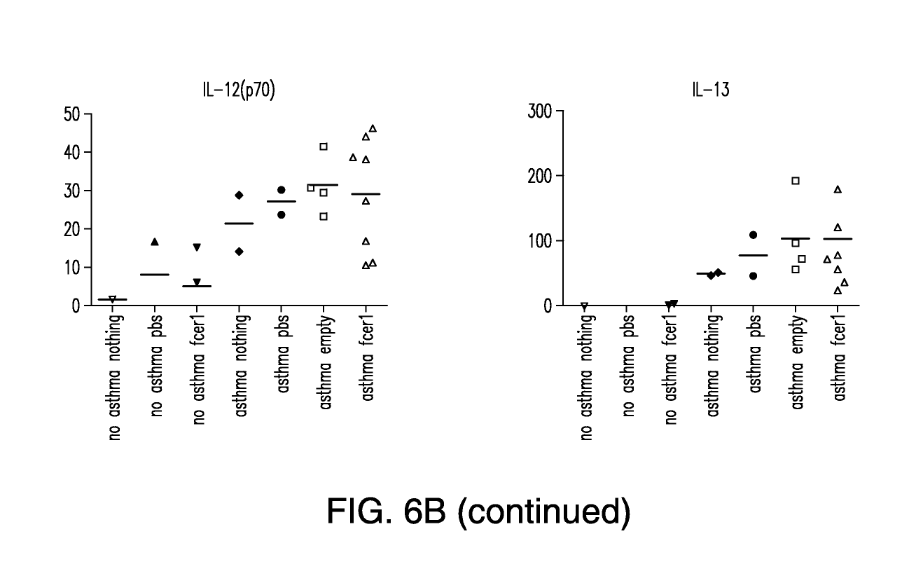

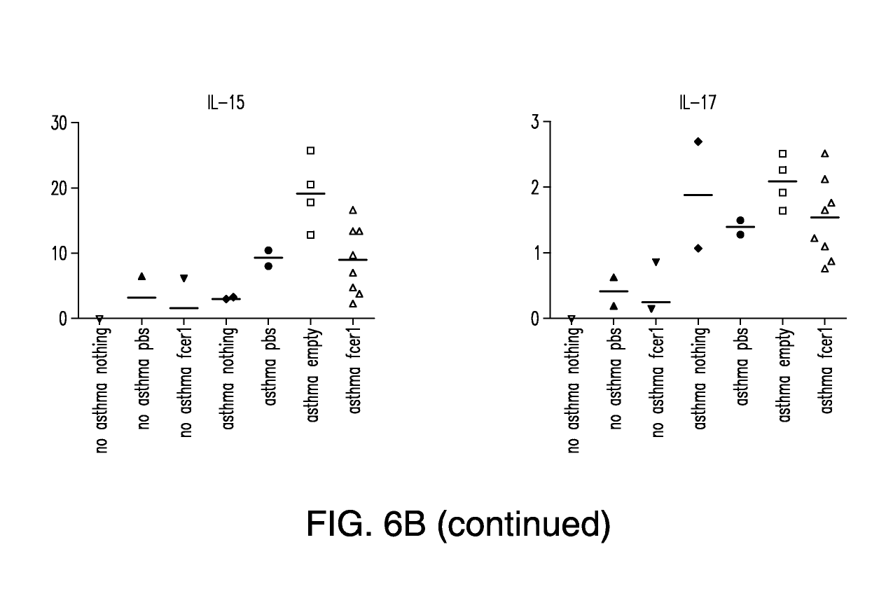

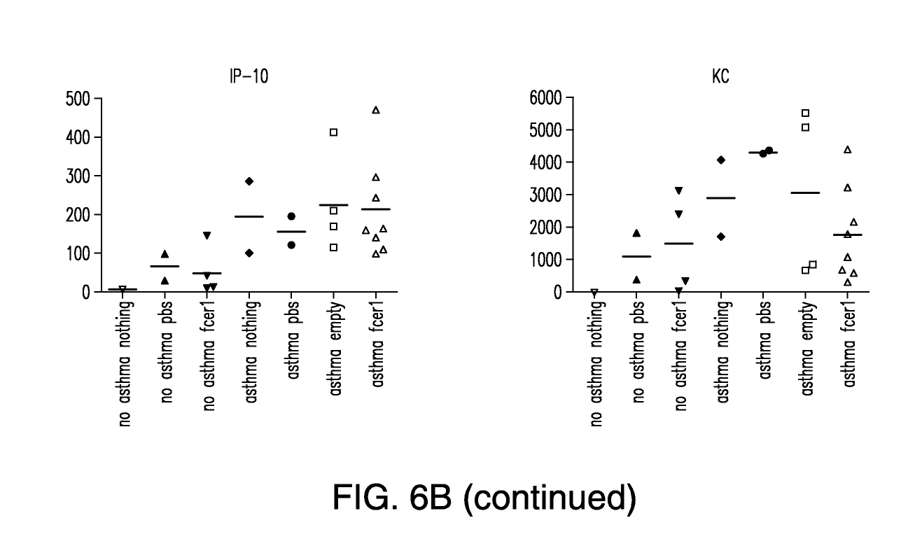

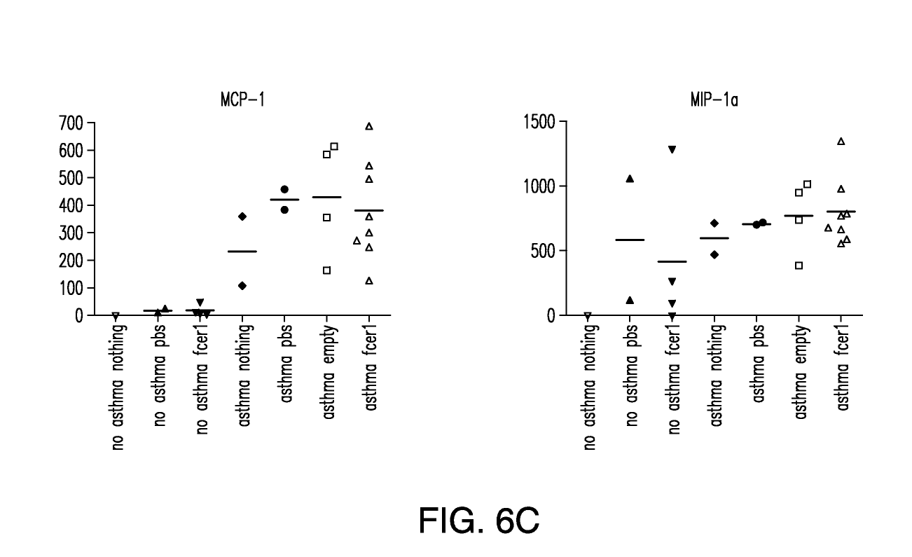

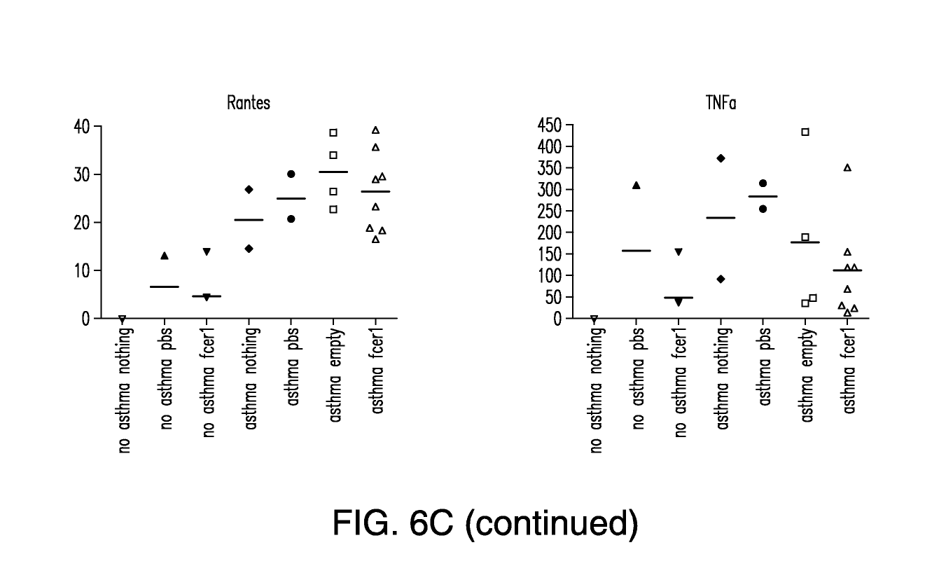

In another series of experiments, the BALF fluid of mice with or without OVA-induced asthma, either treated with FcER1 antisense ODN-loaded microspheres, PBS, or empty microspheres, was tested for the concentrations of various cytokines, including granulocyte colony-stimulating factor (“G-CSF”), granulocyte macrophage colony-stimulating factor (“GM-CSF”), interferon gamma (“IFNg”), interleukin-1beta (“IL-1b”), interleukin-2 (“IL-2”), interleukin-4 (“IL-4”), interleukin-5 (“IL-5”), interleukin-6 (“IL-6”), interleukin-9 (“IL-9”), interleukin-10 (“IL-10”), interleukin-12 (p70) (“IL-12(p70)”), interleukin-13 (“IL-13”), interleukin-15 (“IL-15”), interleukin-17 (“IL-17”), interferon-gamma-induced protein 10 (“IP-10”), keratinocyte-derived cytokine (“KC”), monocyte chemotactic protein-1 (“MCP-1”), macrophage inflammatory protein-1a (“MIP-1a”), the cytokine known as RANTES (for regulated upon activation, normal T cell expressed and secreted), and tumor necrosis factor alpha (“TNFa”). The results are shown in FIG. 6A-C. Of these, administration of the FcER1-loaded microspheres ten days following OVA primary challenge resulted in an apparent decrease in the cytokines GM-CSF, IFNg, IL-9, IL-15, IL-17, KC, and TNFa, and smaller apparent decreases inIL-1b, IL-2, IL-10, IL-12(p70), MCP-1, and RANTES. For other cytokines a smaller decrease, no apparent effect, or an increase was observed. These results show that treatment of asthmatic animals with FcER1-loaded microspheres resulted in the reduction in levels of inflammatory cytokines.

### 7. REFERENCES

1. Boyce, J. A. 2008. Eicosanoids in asthma, allergic inflammation, and host defense. Current molecular medicine 8:335-349.
2. Broide, D. 2008. New perspectives on mechanisms underlying chronic allergic inflammation and asthma in 2007. The Journal of allergy and clinical immunology 122:475-480.
3. Fox, H. 2007. Anti-IgE in severe persistent allergic asthma. Respirology (Carlton, Vic 12 Suppl 3:S22-28; discussion S45-27.
4. Frieri, M. 2007. Advances in the understanding of allergic asthma. Allergy Asthma Proc 28:614-619.
5. Plosker, G. L., and S. J. Keam. 2008. Omalizumab: a review of its use in the treatment of allergic asthma. BioDrugs 22:189-204.
6. Suarez, C. J., N. J. Parker, and P. W. Finn, 2008. Innate immune mechanism in allergic asthma. Current allergy and asthma reports 8:451-459.
7. Verstraelen, S., K. Bloemen, I. Nelissen, H. Witters, G. Schoeters, and R. Van Den Heuvel. 2008. Cell types involved in allergic asthma and their use in in vitro models to assess respiratory sensitization. Toxicol In Vitro 22:1419-1431.
8. Meurs, H., R. Gosens, and J. Zaagsma. 2008. Airway hyperresponsiveness in asthma: lessons from in vitro model systems and animal models. Fur Respir J 32:487-502.
9. Takizawa, H. 2007. Novel strategies for the treatment of asthma. Recent patents on inflammation & allergy drug discovery 1:13-19.
10. Bradding, P., A. F. Walls, and S. T. Holgate. 2006. The role of the mast cell in the pathophysiology of asthma. The Journal of allergy and clinical immunology 117:1277-1284.
11. Hamid, Q., M. K. Tulic, M. C. Liu, and R. Moqbel. 2003. Inflammatory cells in asthma: mechanisms and implications for therapy. The Journal of allergy and clinical immunology 111:S5-S12; discussion S12-17.
12. Leigh, R., R. Ellis, J. Wattie, D. S. Southam, M. De Hoogh, J. Gauldie, P. M. O'Byrne, and M. D. Inman. 2002. Dysfunction and remodeling of the mouse airway persist after resolution of acute allergen-induced airway inflammation. American journal of respiratory cell and molecular biology 27:526-535.
13. Gould, H. J., and B. J, Sutton. 2008. IgE in allergy and asthma today. Nature reviews 8:205-217.
14. Zhang, M., R. F. Murphy, and D. K. Agrawal. 2007. Decoding IgE Fc receptors. Immunol Res 37:1-16,
15. Sanak, M., D. P. Potaczek, E. Nizankowska-Mogilnicka, and A. Szczeklik. 2007. Genetic variability of the high-affinity IgE receptor alpha subunit (Fe epsilon RI alpha) is related to total serum IgE levels in allergic subjects. Allergol Int 56:397-401.
16. Kraft, S., and J. P. Kinet. 2007. New developments in FcepsilonRl regulation, function and inhibition. Nature reviews 7:365-378.
17. Klemm, S., and J. Ruland. 2006. Inflammatory signal transduction from the Fc epsilon RI to NF-kappa B. Immunobiology 211:815-820.
18. Poole, J. A., and L. J. Rosenwasser. 2005. The role of immunoglobulin E and immune inflammation: implications in allergic rhinitis. Current allergy and asthma reports 5:252-258.
19. Poole, J. A., P. Matangkasombut, and L. J. Rosenwasser. 2005. Targeting the IgE molecule in allergic and asthmatic diseases: review of the IgE molecule and clinical efficacy. The Journal of allergy and clinical immunology 115:S376-385.
20. MacGlashan, D., Jr. 2005. IgE and FcepsilonRl regulation. Clin Rev Allergy Immunol 29:49-60.
21. Conner, E. R., and S. S. Saini. 2005. The immunoglobulin E receptor: expression and regulation. Current allergy and asthma reports 5:191-196.
22. Stokes, J., and T. B. Casale. 2004. Rationale for new treatments aimed at IgE immunomodulation. Ann Allergy Asthma Immunol 93:212-217; quiz 217-219, 271.
23, von Bubnoff, D., N. Novak, S. Kraft, and T. Bieber. 2003. The central role of FcepsilonRl in allergy. Clinical and experimental dermatology 28:184-187.
24. Rivera, J., and A. Olivera. 2008. A current understanding of Fe epsilon RI-dependent mast cell activation. Current allergy and asthma reports 8:14-20.
25. Brown, J. M., T. M. Wilson, and D. D. Metcalfe. 2008. The mast cell and allergic diseases: role in pathogenesis and implications for therapy. Clin Exp Allergy 38:4-18.
26. Theoharides, T. C., D. Kempuraj, M. Tagen, P. Conti, and D. Kalogeromitros. 2007. Differential release of mast cell mediators and the pathogenesis of inflammation. Immunol Rev 217:65-78.

27. Doherty, T., and D. Broide. 2007. Cytokines and growth factors in airway remodeling in asthma. Curr Opin Immunol 19:676-680.
28. Gibbs, B. F., A. Rathling, D. Zillikens, M. Huber, and H. Haas. 2006. Initial Fe epsilon RI-mediated signal strength plays a key role in regulating basophil signaling and deactivation. The Journal of allergy and clinical immunology 118:1060-1067.
29. Peachell, P. 2005. Targeting the mast cell in asthma. Current opinion in pharmacology 5:251-256.
30. Falcone, F. H., D. Zillikens, and B. F. Gibbs. 2006. The 21st century renaissance of the basophil? Current insights into its role in allergic responses and innate immunity. Experimental dermatology 15:855-864.
31. Shum, B. O., M. S. Rolph, and W. A. Sewell. 2008. Mechanisms in allergic airway inflammation—lessons from studies in the mouse. Expert Rev Mol Med 10:e15.
32. McCloskey, N., J. Hunt, R. L. Beavil, M. R. Jutton, G. J. Grundy, E. Girardi, S. M. Fabiane, D. J. Fear, D. H. Conrad, B. J. Sutton, and H. J. Gould. 2007. Soluble CD23 monomers inhibit and oligomers stimulate TGE synthesis in human B cells. J Biol Chem 282:24083-24091.
33. Ford, J. W., M. A. Kilmon, K. M. Haas, A. E. Shelburne, Y. Chan-Li, and D. H. Conrad. 2006. In vivo murine CD23 destabilization enhances CD23 shedding and IgE synthesis. Cellular immunology 243:107-117.
34. Chang, T. W. 2006. Developing antibodies for targeting immunoglobulin and membrane-bound immunoglobulin E. Allergy Asthma Proc 27:S7-14,
35. Tu, Y., S. Salim, J. Bourgeois, V. Di Leo, E. J. Irvine, J. K. Marshall, and M. H. Perdue. 2005. CD23-mediated IgE transport across human intestinal epithelium: inhibition by blocking sites of translation or binding. Gastroenterology 129:928-940.
36. Rosenwasser, L. J., and J. Meng. 2005. Anti-CD23. Clin Rev Allergy Immunol 29:61-72.

37. Poole, J. A., J. Meng, M. Reff, M. C. Spellman, and L. J. Rosenwasser, 2005. Anti-CD23 monoclonal antibody, lumiliximab, inhibited allergen-induced responses in antigen-presenting cells and T cells from atopic subjects. The Journal of allergy and clinical immunology 116:780-788.
38. Holgate, S., T. Casale, S. Wenzel, J. Bousquet, Y. Deniz, and C. Reisner. 2005. The anti-inflammatory effects of omalizumab confirm the central role of IgE in allergic inflammation. The Journal of allergy and clinical immunology 115:459-465.
39. Hibbert, R. G., P. Teriete, G. J. Grundy, R. L. Beavil, R. Reljic, V. M. Holers, J. P. Hannan, B. J. Sutton, H. J. Gould, and J. M. McDonnell. 2005. The structure of human CD23 and its interactions with IgE and CD21. J Exp Med 202:751-760.
40. Belleau, J. T., R. K. Gandhi, H. M. McPherson, and D. B. Lew. 2005. Research upregulation of CD23 (FcepsilonRII) expression in human airway smooth muscle cells (huASMC) in response to IL-4, GM-CSF, and IL-4/GM-CSF. Clin Mol Allergy 3:6.
41. Amrol, D. J., D. D. Hagaman, J. R. Sheller, and J. J. Murray, 2005. Soluble CD23 and interleukin-1 receptor antagonist in human asthmatics following antigen challenge. J Asthma 42:73-76.
42. Reichert, J. M. 2004. Technology evaluation: lumiliximab, Biogen Idec. Current opinion in molecular therapeutics 6:675-683.
43. Ayres, J. G., B. Higgins, E. R. Chilvers, G. Ayre, M. Blogg, and H. Fox. 2004. Efficacy and tolerability of anti-immunoglobulin E therapy with omalizumab in patients with poorly controlled (moderate-to-severe) allergic asthma. Allergy 59:701-708.
44. Edwards, M. R., N. W. Bartlett, D. Clarke, M. Birrell, M. Belvisi, and S. L. Johnston. 2009. Targeting the NF-kappaB pathway in asthma and chronic obstructive pulmonary disease. Pharmacology & therapeutics 121:1-13.
45. Tversky, J. R., T. V. Le, A. P. Bieneman, K. L. Chichester, R. G. Hamilton, and J. T. Schroeder. 2008. Human blood dendritic cells from allergic subjects have impaired capacity to produce interferon-alpha via Toll-like receptor 9. Clin Exp Allergy 38:781-788.

46. Catley, M. C., J. E. Chivers, N. S. Holden, P. J. Barnes, and R. Newton. 2005. Validation of IKK beta as therapeutic target in airway inflammatory disease by adenoviral-mediated delivery of dominant-negative IKK beta to pulmonary epithelial cells. British journal of pharmacology 145:114-122.
47. Birrell, M. A., E. Hardaker, S. Wong, K. McCluskie, M. Catley, J. De Alba, R. Newton, S. Haj-Yahia, K. T. Pun, C. J. Watts, R. J. Shaw, T. J. Savage, and M. G. Belvisi. 2005. Ikappa-B kinase-2 inhibitor blocks inflammation in human airway smooth muscle and a rat model of asthma. American journal of respiratory and critical care medicine 172:962-971.
48. Bellou, A., and P. W. Finn, 2005. Costimulation: critical pathways in the immunologic regulation of asthma. Current allergy and asthma reports 5:149-154.
49. Beeren, I. M., M. S. De Bruin-Weller, C. Ra, I. Kok, C. A. Bruinzeel-Koomen, and E. F. Knol. 2005. Expression of Fc(epsilon)RI on dendritic cell subsets in peripheral blood of patients with atopic dermatitis and allergic asthma. The Journal of allergy and clinical immunology 116:228-229.
50. McMillan, S. J., and C. M. Lloyd. 2004. Prolonged allergen challenge in mice leads to persistent airway remodelling. Clin Exp Allergy 34:497-507.
51. Maurer, D., S. Fiebiger, C. Ebner, B. Reininger, G. F. Fischer, S. Wichlas, M. H. Jouvin, M. Schmitt-Egenolf, D. Kraft, J. P. Kinet, and G. Stingl. 1996. Peripheral blood dendritic cells express Fc epsilon RI as a complex composed of Fc epsilon RI alpha- and Fc epsilon RI gamma-chains and can use this receptor for IgE-mediated allergen presentation. J Immunol 157:607-616.
52. Bruhns, P., S. Fremont, and M. Daeron. 2005. Regulation of allergy by Fc receptors. Curr Opin Immunol 17:662-669.
53. Metzger, H. 1992. The receptor with high affinity for IgE. Immunol Rev 125:37-48.
54. Ishizaka, K. 1989. Basic mechanisms of IgE-mediated hypersensitivity. Curr Opin Immunol 1:625-629.
55. Conrad, D. H., J. W. Ford, J. L. Sturgill, and D. R. Gibb. 2007. CD23: an overlooked regulator of allergic disease. Current allergy and asthma reports 7:331-337.
56. Pongratz, G., J. W. McAlees, D. H. Conrad, R. S. Erbe, K. M. Haas, and V. M. Sanders. 2006. The level of IgE produced by a B cell is regulated by norepinephrine in a p38 MAPK- and CD23-dependent manner. J Immunol 177:2926-2938.
57. Barnes, P. J. 2006. Transcription factors in airway diseases. Laboratory investigation; a journal of technical methods and pathology 86:867-872.
58. Desmet, C., P. Gosset, B. Pajak, D. Cataldo, M. Bentires-Alj, P. Lekeux, and F. Bureau. 2004. Selective blockade of NF-kappa B activity in airway immune cells inhibits the effector phase of experimental asthma. J Immunol 173:5766-5775.
59. Choi, I. W., D. K. Kim, H. M. Ko, and H. K. Lee. 2004. Administration of antisense phosphorothioate oligonucleotide to the p65 subunit of NF-kappaB inhibits established asthmatic reaction in mice. International immunopharmacology 4:1817-1828.
60. Wegener, E., and D. Krappmann. 2008. Dynamic protein complexes regulate NF-kappaB signaling. Handbook of experimental pharmacology:237-259.
61. Simmonds, R. E., and B. M. Foxwell. 2008. Signalling, inflammation and arthritis: NF-kappaB and its relevance to arthritis and inflammation. Rheumatology (Oxford, England) 47:584-590.
62. Sarkar, F. H., Y. Li, Z. Wang, and D, Kong. 2008. NF-kappaB signaling pathway and its therapeutic implications in human diseases. International reviews of immunology 27:293-319.
63. Ghosh, S., and M. S. Hayden. 2008. New regulators of NF-kappaB in inflammation. Nature reviews 8:837-848.
64. Basak, S., and A. Hoffmann. 2008. Crosstalk via the NF-kappaB signaling system. Cytokine & growth factor reviews 19:187-197.
65. Nam, N. H. 2006. Naturally occurring NF-kappaB inhibitors. Mini reviews in medicinal chemistry 6:945-951.
66. Ennis, M. 2006. New targets for modifying mast cell activation in asthma. Current allergy and asthma reports 6:247-251.
67. Adcock, I. M., K. F. Chung, G. Caramori, and K. Ito. 2006. Kinase inhibitors and airway inflammation. European journal of pharmacology 533:118-132.
68. Peng, Y., M. R. Power, B. Li, and T. J. Lin. 2005. Inhibition of IKK down-regulates antigen+IgE-induced TNF production by mast cells: a role for the IKK-IkappaB-NF-kappaB pathway in IgE-dependent mast cell activation. Journal of leukocyte biology 77:975-983.
69. Kim, H. M., K. S. Kim, and E. H. Lee. 1998. Specific inhibition of immunoglobulin E-mediated allergic reaction using antisense Fe epsilon RI alpha oligodeoxynucleotides. Immunology 93:589-594.
70. Ryan, J. J., C. A. Kinzer, and W. E. Paul. 1995. Mast cells lacking the high affinity immunoglobulin E receptor are deficient in Fe epsilon RI gamma messenger RNA. J Exp Med 182:567-574.
71. Haak-Frendscho, M., J. Ridgway, R. Shields, K. Robbins, C. Gorman, and P. Jardieu, 1993. Human IgE receptor alpha-chain IgG chimera blocks passive cutaneous anaphylaxis reaction in vivo, J Immunol 151:351-358.
72. Hall, T. J., and J. Brostoff. 1992. Specific inhibition of IgE antibody production by an antisense oligodeoxynucleotide oligomer (Oligostick). Immunology 77:462-464.
73. Ball, H. A., A. Sandrasagra, L. Tang, M. Van Scott, J. Wild, and J. W. Nyce. 2003. Clinical potential of respirable antisense oligonucleotides (RASONs) in asthma. Am J Pharmacogenomics 3:97-106.
74. Sandrasagra, A., S. A. Leonard, L. Tang, K. Teng, Y, Li, H. A. Ball, J. C. Mannion, and J. W. Nyce. 2002. Discovery and development of respirable antisense therapeutics for asthma. Antisense & nucleic acid drug development 12:177-181.
75. Tanaka, M., and J. W. Nyce, 2001. Respirable antisense oligonucleotides: a new drug class for respiratory disease. Respiratory research 2:5-9.
76. Sandrasagra, A., L. Tang, S, A. Leonard, K. Teng, Y. Li, J. C. Mannion, and J. W. Nyce. 2001. RASONs: a novel antisense oligonucleotide therapeutic approach for asthma. Expert opinion on biological therapy 1:979-983.
77. Ali, S., S. A. Leonard, C. A. Kukoly, W. J. Metzger, W. R. Wooles, J. F. McGinty, M. Tanaka, A. Sandrasagra, and J. W. Nyce. 2001. Absorption, distribution, metabolism, and excretion of a respirable antisense oligonucleotide for asthma. American journal of respiratory and critical care medicine 163:989-993.
78. Metzger, W. J., and J. W. Nyce. 1999. Respirable Antisense Oligonucleotide (RASON) Therapy for Allergic Asthma. BioDrugs 12:237-243.
79. Joseph, M., A. S. Gounni, J. P. Kusnierz, H. Vorng, M. Sarfati, J. P. Kinet, A. B. Tonnel, A. Capron, and M. Capron. 1997. Expression and functions of the high-affinity IgE receptor on human platelets and megakaryocyte precursors. European journal of immunology 27:2212-2218.
80. Kovacs, J. R., J. Tidball, A. Ross, L. Jia, Y. Zheng, E. S. Gawalt, and W. S. Meng. 2009. Characterization of nickel-decorated PLGA particles anchored with a his-tagged polycation. Journal of biomaterials science 20:1307-1320.
81. Chalk, A. M., and E. L. Sonnhammer. 2008. siRNA specificity searching incorporating mismatch tolerance data. Bioinformatics (Oxford, England) 24:1316-1317.
82. Iyer, S., K. Deutsch, X. Yan, and B. Lin, 2007. Batch RNAi selector: a standalone program to predict specific siRNA candidates in batches with enhanced sensitivity. Computer methods and programs in biomedicine 85:203-209.
83. Ryan, M. C., B. R. Zeeberg, N. J. Caplen, J. A. Cleland, A. B. Kahn, H. Liu, and J. N. Weinstein. 2008, SpliceCenter: a suite of web-based bioinformatic applications for evaluating the impact of alternative splicing on RT-PCR, RNAi, microarray, and peptide-based studies. BMC bioinformatics 9:313.
84. Shah, J. K., H. R. Garner, M. A. White, D. S. Shames, and J. D. Minna. 2007. sIR: siRNA Information Resource, a web-based tool for siRNA sequence design and analysis and an open access siRNA database. BMC bioinforrnatics 8:178.
85. Arrigo, P., P. Scartezzini, and P. Romano. 2002. AgeWa: an integrated approach for antisense experiment design. IEEE transactions on nanobioscience 1:167-171.
86. Li, Y. Y., L. Qin, Z. M. Guo, L. Liu, H. Xu, P. Hao, J. Su, Y. Shi, W. Z. He, and Y. X. Li. 2006. In silico discovery of human natural antisense transcripts. BMC bioinformatics 7:18,
87. Lu, Z. J., and D. H. Mathews. 2008. Fundamental differences in the equilibrium considerations for siRNA and antisense oligodeoxynucleotide design. Nucleic Acids Res 36:3738-3745.
88. Anderson, E. M., A. Birmingham, S. Baskerville, A. Reynolds, E. Maksimova, D. Leake, Y. Fedorov, J. Karpilow, and A. Khvorova. 2008. Experimental validation of the importance of seed complement frequency to siRNA specificity. RNA (New York, N.Y. 14:853-861.
89. Birmingham, A., E. M. Anderson, A. Reynolds, D. Ilsley-Tyree, D. Leake, Y. Fedorov, S. Baskerville, E. Maksimova, K. Robinson, J. Karpilow, W. S. Marshall, and A. Khvorova. 2006. 3′ UTR seed matches, but not overall identity, are associated with RNAi off-targets. Nature methods 3:199-204.
90. Ericson, E., M. Gebbia, L. E. Heisler, J. Wildenhain, M. Tyers, G. Giaever, and C. Nislow. 2008. Off-target effects of psychoactive drugs revealed by genome-wide assays in yeast. PLoS genetics 4:e1000151.
91. Vankoningsloo, S., F. de Longueville, S. Evrard, P. Rahier, A. Houbion, A. Fattaccioli, M. Gastellier, J. Remade, M. Raes, P. Renard, and T. Arnould. 2008. Gene expression silencing with ‘specific’ small interfering RNA goes beyond specificity—a study of key parameters to take into account in the onset of small interfering RNA off-target effects. The REBS journal 275:2738-2753.
92. El Bakkouri, K., A. Wullaert, M. Haegman, K. Heyninek, and R. Beyaert. 2005. Adenoviral gene transfer of the NF-kappa B inhibitory protein ABIN-1 decreases allergic airway inflammation in a murine asthma model, J Biol Chem 280:17938-17944.
93. Furumoto, Y., S. Nunomura, T. Terada, J. Rivera, and C. Ra. 2004. The FcepsilonRlbeta immunoreceptor tyrosine-based activation motif exerts inhibitory control on MAPK and IkappaB kinase phosphorylation and mast cell cytokine production. J Biol Chem 279:49177-49187.
94. Gurish, M. F., N. Ghildyal, J. Arm, K. F. Austen, S. Avraham, D. Reynolds, and R. L. Stevens. 1991. Cytokine mRNA are preferentially increased relative to secretory granule protein mRNA in mouse bone marrow-derived mast cells that have undergone IgE-mediated activation and degranulation. J Immunol 146:1527-1533.
95. Holden, N. S., W. Gong, E. M. King, M, Kaur, M. A. Giembycz, and R. Newton. 2007. Potentiation of NF-kappaB-dependent transcription and inflammatory mediator release by histamine in human airway epithelial cells. British journal of pharmacology 152:891-902.
96. Kim, K., Y. Kim, H. Y. Kim, J. Y. Ro, and D. Jeoung, 2008. Inhibitory mechanism of anti-allergic peptides in RBL2H3 cells. European journal of pharmacology 581:191-203.
97. Oliver-Vila, I., I. Saborit-Villarroya, P. Engel, and M. Martin. 2008. The leukocyte receptor CD84 inhibits Fe epsilon RI-mediated signaling through hemophilic interaction in transfected RBL-2H3 cells. Molecular immunology 45:2138-2149.
98. Cates, E. C., R. Fattouh, J. Wattie, M. D. Inman, S. Goncharova, A. J. Coyle, J. C. Gutierrez-Ramos, and M. Jordana. 2004. Intranasal exposure of mice to house dust mite elicits allergic airway inflammation via a GM-CSF-mediated mechanism. J Immunol 173:6384-6392.
99. Gajewska, B. U., D. Alvarez, M. Vidric, S. Goncharova, M. R. Stampfli, A. J. Coyle, J. C. Gutierrez-Ramos, and M. Jordana. 2001. Generation of experimental allergic airways inflammation in the absence of draining lymph nodes. The Journal of clinical investigation 108:577-583.
100. Gajewska, B. U., F. K. Swirski, D. Alvarez, S. A. Ritz, S. Goncharova, M. Cundall, D. P. Snider, A. J. Coyle, J. C. Gutierrez-Ramos, M. R. Stampfli, and M. Jordana. 2001. Temporal-spatial analysis of the immune response in a murine model of ovalbumin-induced airways inflammation. American journal of respiratory cell and molecular biology 25:326-334.
101. Johnson, J. R., R. E. Wiley, R. Fattouh, F. K. Swirski, B. U. Gajewska, A. J. Coyle, J. C. Gutierrez-Ramos, R. Ellis, M. D. Inman, and M. Jordana. 2004. Continuous exposure to house dust mite elicits chronic airway inflammation and structural remodeling. American journal of respiratory and critical care medicine 169:378-385.
102. Lei, X. F., Y. Ohkawara, M. R. Stampfli, J. Gauldie, K. Croitoru, M. Jordana, and Z. Xing. 1998. Compartmentalized transgene expression of granulocyte-macrophage colony-stimulating factor (GM-CSF) in mouse lung enhances allergic airways inflammation. Clinical and experimental immunology 113:157-165.
103. Leigh, R., D. Vethanayagam, M. Yoshida, R. M, Watson, T. Rerecich, M. D. Inman, and P. M. O'Byrne. 2002. Effects of montelukast and budesonide on airway responses and airway inflammation in asthma. American journal of respiratory and critical care medicine 166:1212-1217.
104. Stampfli, M. R., R. E. Wiley, G. S. Neigh, B. U. Gajewska, X. F. Lei, D. P. Snider, Z. Xing, and M. Jordana. 1998. GM-CSF transgene expression in the airway allows aerosolized ovalbumin to induce allergic sensitization in mice. The Journal of clinical investigation 102:1704-1714.
105. Swirski, F. K., 13. U. Gajewska, C. S. Robbins, A. D'Sa, J. R. Johnson, M. A. Pouladi, M. D. Inman, and M. R. Stampfli. 2004. Concomitant airway expression of granulocyte-macrophage colony-stimulating factor and decorin, a natural inhibitor of transforming growth factor-beta, breaks established inhalation tolerance. European journal of immunology 34:2375-2386.
106. Wiley, R., K. Palmer, B. Gajewska, M. Stamp D. Alvarez, A. Coyle, J. Gutierrez-Ramos, and M. Jordana. 2001. Expression of the Th1 chemokine IFN-gamma-inducible protein 10 in the airway alters mucosal allergic sensitization in mice. J Immunol 166:2750-2759.
107. Isenberg-Feig, H., J. P. Justice, and A. Keane-Myers. 2003. Animal models of allergic asthma. Current allergy and asthma reports 3:70-78.

108. Lloyd, C. M. 2007. Building better mouse models of asthma. Current allergy and asthma reports 7:231-236.
109. Fuchs, B., and A. Braun. 2008. Improved mouse models of allergy and allergic asthma—chances beyond ovalbumin. Curr Drug Targets 9:495-502.
110. Hamelmann, E., A. Oshiba, J. Schwarze, K. Bradley, J. Loader, G. L. Larsen, and E. W. Gelfand. 1997. Allergen-specific IgE and IL-5 are essential for the development of airway hyperresponsiveness. American journal of respiratory cell and molecular biology 16:674-682.
111. Hamelmann, E., J. Schwarze, K. Takeda, A. Oshiba, G. L. Larsen, C. G. Irvin, and E. W. Gelfand. 1997. Noninvasive measurement of airway responsiveness in allergic mice using barometric plethysmography. American journal of respiratory and critical care medicine 156:766-775.
112. Schwarze, J., E. Hamelmann, K. L. Bradley, K. Takeda, and E. W. Gelfand. 1997. Respiratory syncytial virus infection results in airway hyperresponsiveness and enhanced airway sensitization to allergen. The Journal of clinical investigation 100:226-233.
113. Ewart, S., R. Levitt, and W. Mitzner. 1995. Respiratory system mechanics in mice measured by end-inflation occlusion. J Appl Physiol 79:560-566.
114. Ewart, S., R. Levitt, and W. Mitzner. 2001. Ventilation in mice with a solenoid-controlled airflow. J Appl Physiol 90:2016-2017.
115. Ewart, S. L., W. Mitzner, D. A. DiSilvestre, D. A. Meyers, and R. C. Levitt. 1996. Airway hyperresponsiveness to acetylcholine: segregation analysis and evidence for linkage to murine chromosome 6. American journal of respiratory cell and molecular biology 14:487-495.
116. Inman, M. D., R. Ellis, J. Wattie, J. A. Denburg, and P. M. O'Byrne. 1999. Allergen-induced increase in airway responsiveness, airway eosinophilia, and bone-marrow eosinophil progenitors in mice. American journal of respiratory cell and molecular biology 21:473-479.

117. Tankersley, C. G., R. S. Fitzgerald, R. C. Levitt, W. A. Mitzner, S. L. Ewart, and S. R. Kleeberger. 1997. Genetic control of differential baseline breathing pattern. J Appl Physiol 82:874-881.
118. Volgyesi, G. A., L. N. Tremblay, P. Webster, N. Zamel, and A. S. Slutsky. 2000. A new ventilator for monitoring lung mechanics in small animals. J Appl Physiol 89:413-421.
119. U.S. Pat. No. 5,892,023.

120. U.S. Pat. No. 7,101,851.

121. U.S. Pat. No. 6,699,472.

122. U.S. Pat. No. 6,685,939.

123. U.S. Pat. No. 5,965,709.

124. International Patent Application Publication No, WO2005/112894.

125. International Patent Application Publication No. WO2005/112885.

126. United States Patent Application Publication No. US2006/0024240A1.

127. United States Patent Application Publication No. US2008/0039369A1.

128. International Patent Application No. PCT/US2009/041167.

| SEQ ID NO: 1 = NFKAPPAB Consensus binding sequence | |
|---|---|
| 5′CTGGGGACTTTCCAT-3′ | |
| SEQ ID NO: 2 = complement of SEQ ID NO: 1 | |
| 5′ ATGGAAAGTCCCCAG 3′. | |
| SEQ ID NO: 3 = Homologous FcERI-alpha (human/mouse from 3′ end of sequence): | |
| gtg tcc aca gca aac aga atc | |
| SEQ ID NO: 4 = cationic peptide | |
| Orn-Orn-Orn-Orn-Orn-Orn-Orn-Orn-Orn-Orn-His-His-His-His-His-His | |
| SEQ ID NO: 5 = human alpha subunit FcERI = ncbi Ref. No. NM_002001.2 | |
| 1 | agtctccagc atcctccacc tgtctaccac cgagcatggg cctatatttg aagccttaga |
| 61 | tctctccagc acagtaagca ccaggagtcc atgaagaaga tggctcctgc catggaatcc |
| 121 | cctactctac tgtgtgtagc cttactgttc ttcgctccag atggcgtgtt agcagtccct |
| 181 | cagaaaccta aggtctcctt gaaccctcca tggaatagaa tatttaaagg agagaatgtg |
| 241 | actcttacat gtaatgggaa caatttcttt gaagtcagtt ccaccaaatg gttccacaat |
| 301 | ggcagccttt cagaagagac aaattcaagt ttgaatattg tgaatgccaa atttgaagac |
| 361 | agtggagaat acaaatgtca gcaccaacaa gttaatgaga gtgaacctgt gtacctggaa |
| 421 | gtcttcagtg actggctgct ccttcaggcc tctgctgagg tggtgatgga gggccagccc |
| 481 | ctcttcctca ggtgccatgg ttggaggaac tgggatgtgt acaaggtgat ctattataag |
| 541 | gatggtgaag ctctcaagta ctggtatgag aaccacaaca tctccattac aaatgccaca |
| 601 | gttgaagaca gtggaaccta ctactgtacg ggcaaagtgt ggcagctgga ctatgagtct |
| 661 | gagcccctca acattactgt aataaaagct ccgcgtgaga agtactggct acaatttttt |
| 721 | actcaattgt tggtggtgat tctgtttgct gtggacacag gattatttat ctcaactcag |
| 781 | cagcaggtca catttctctt gaagattaag agaaccagga aaggcttcag acttctgaac |
| 841 | ccacatccta agccaaaccc caaaaacaac tgatataatt actcaagaaa tatttgcaac |
| 901 | attagttttt ttccagcatc agcaattgct actcaattgt caaacacagc ttgcaatata |
| 961 | catagaaacg tctgtgctca aggatttata gaaatgcttc attaaactga gtgaaactgg |
| 1021 | ttaagtggca tgtaatagta agtgctcaat taacattggt tgaataaatg agagaatgaa |
| 1081 | tagattcatt tattagcatt tgtaaaagag atgttcaatt tcaataaaat aaatataaaa |
| 1141 | ccatgtaaca gaatgcttct gagtaaaaaa aaaaaaaaaa aaaaaaaaaa a |
| SEQ ID NO: 6 = murine alpha subunit FcERI = NCBI Acc. No. NM_010184 | |
| 1 | tattttcgaa gccatagctc tctggtgcag ttagcacctg aaggtgcagg ggcgatggtc |
| 61 | actggaatgt ctgcccagct gtgcctagca ctgctgttca tgtctcttga tgtcattctg |
| 121 | acagccactg agaaatctgt actgaccttg gacccaccat ggattagaat atttacagga |
| 181 | gagaaagtga ccctttcctg ctatgggaac aatcaccttc aaatgaactc tactactaaa |
| 241 | tggatccaca atggtaccgt ctctgaggtg aactcttcac atttggtcat tgtgagtgcc |
| 301 | accgttcaag acagtggaaa atacatatgt cagaagcaag gattgtttaa gagtaaacct |
| 361 | gtgtacttga atgtaacgca agattggctg ctccttcaga catctgctga catggtctta |
| 421 | gtccatggat cctttgacat cagatgccat ggctggaaga actggaatgt ccgcaaggtg |
| 481 | atctactaca ggaatgacca tgctttcaac tacagttatg agagccccgt ctccattaga |
| 541 | gaggccacac tgaatgacag tggcacctac cactgcaagg gctatcttag gcaggtgaaa |
| 601 | tatgaatctg acaaattcag aattgctgta gtaaaagctt acaaatgcaa gtattattgg |
| 661 | ctacaactaa ttttcccatt gttggtggcg attctgtttg ctgtggacac ggggttattg |
| 721 | ctctcaaccg aagaacagtt caaatcagtc ttggagattc agaagactgg aaaatacaag |
| 781 | aaagttgaaa ccgaactcct aacctagata catataggaa ctaatgtcat tgctgaagaa |
| 841 | gcaattctta acagtaattt tctgcagaat cctcaatagc ttctccactg tcaaaggcca |
| 901 | ctcatgtgat ccctagaaaa gtctgttcca caggaattgc ataaatgctt cattaaacca |
| 961 | actgcagctg gttatgtaac atgcaataaa tagcaaatac tcaataaaca ctgccttaat |
| 1021 | aagtta |
| SEQ ID NO: 7 = human beta subunit FcERI = NCBI Acc No. NM_000139.4 = | |
| 1 | aacccatttc aactgcctat tcagagcatg cagtaagagg aaatccacca agtctcaata |
| 61 | taataatatt ctttattcct ggacagctcg gttaatgaaa aaatggacac agaaagtaat |
| 121 | aggagagcaa atcttgctct cccacaggag ccttccagtg tgcctgcatt tgaagtcttg |
| 181 | gaaatatctc cccaggaagt atcttcaggc agactattga agtcggcctc atccccacca |
| 241 | ctgcatacat ggctgacagt tttgaaaaaa gagcaggagt tcctgggggt aacacaaatt |
| 301 | ctgactgcta tgatatgcct ttgttttgga acagttgtct gctctgtact tgatatttca |
| 361 | cacattgagg gagacatttt ttcatcattt aaagcaggtt atccattctg gggagccata |
| 421 | tttttttcta tttctggaat gttgtcaatt atatctgaaa ggagaaatgc aacatatctg |
| 481 | gtgagaggaa gcctgggagc aaacactgcc agcagcatag ctgggggaac gggaattacc |
| 541 | atcctgatca tcaacctgaa gaagagcttg gcctatatcc acatccacag ttgccagaaa |
| 601 | ttttttgaga ccaagtgctt tatggcttcc ttttccactg aaattgtagt gatgatgctg |
| 661 | tttctcacca ttctgggact tggtagtgct gtgtcactca caatctgtgg agctggggaa |
| 721 | gaactcaaag gaaacaaggt tccagaggat cgtgtttatg aagaattaaa catatattca |
| 781 | gctacttaca gtgagttgga agacccaggg gaaatgtctc ctcccattga tttataagaa |
| 841 | tcacgtgtcc agaacactct gattcacagc caaggatcca gaaggccaag gtcttgttaa |
| 901 | ggggctactg gaaaaatttc tattctctcc acagcctgct ggttttacat tagatttatt |
| 961 | cgcctgataa gaatattttg tttctgctgc ttctgtccac cttaatattc tccttctatt |
| 1021 | tgtagatatg atagactcct atttttcttg ttttatatta tgaccacaca catctctgct |
| 1081 | ggaaagtcaa catgtagtaa gcaagattta actgtttgat tataactgtg caaatacaga |
| 1141 | aaaaaagaag gctggctgaa agttgagtta aactttgaca gtttgataat atttggttct |
| 1201 | tagggttttt tttttttttt agcattctta atagttacag ttgggcatga tttgtaccat |
| 1261 | ccacccatac ccacacagtc acagtcacac acacatatgt attacttaca ctatatataa |
| 1321 | cttcctatgc aaatatttta ccaccagtca ataatacatt tttgccaaga catgaagttt |
| 1381 | tataaagatc tgtataattg cctgaatcac cagcacattc actgacatga tattatttgc |
| 1441 | agattgacaa gtaggaagtg gggaatttta ttaagttact cgttgtctgg ggaggtaaat |
| 1501 | aggttaaaaa cagggaaatt ataagtgcag agattaacat ttcacaaatg tttagtgaaa |
| 1561 | catttgtgaa aaaagaagac taaattaaga cctgagctga aataaagtga gtggaaatgg |
| 1621 | aaataatggt tatatctaaa acatgtagaa aaagagtaac tggtagattt tgttaacaaa |
| 1681 | ttaaagaata aagttagaca agcaactggt tgactaatac attaagcgtt tgagtctaag |
| 1741 | atgaaaggag aacactggtt atgttgatag aatgataaaa agggtcgggc gcggaggctc |
| 1801 | acgcctgtaa tcccagccct ttgggaggcc gaggtgggca gatcacgaag tcagtagttt |
| 1861 | gagaccagcc tggccaacat agtgaaaccc cgtctceact aaaaatacaa aaaaaaaatt |
| 1921 | agctgggtgt ggtggcagtc acctgtagtc ccagctactt gggaggctga ggcaggagaa |
| 1981 | tcgcttcaac ctgggaggcg gaggttgcag tgagccgaga tcgcaccagt gcactccagc |
| 2041 | cttggtgaca atgggagact ccatctcaaa aaaaaaaaaa aaaaaaaaaa agataaaaag |
| 2101 | tcagaaatct gaaaagtgga ggaagagtac aaatagacct aaattaagct catttttagg |
| 2161 | ctttgatttt ggggagacaa agggaaatgc agccatagag ggcctgatga catccaatac |
| 2221 | agagttctgg taaagataaa atttgataca ggtttggtgt cattataaga gaaatcatta |
| 2281 | ttaaatgaag caagttaaca ctctaagaga attattttga gatagaagtg aagctaagct |
| 2341 | aaacttcaca tgcctataat tggagggaaa aactaaggat aaaatctagc ctagaagata |
| 2401 | caataattag tcataaacat gcattgtgaa actgtagaga gcaggtagcc caaaatagag |
| 2461 | aaagattaga taaagagaaa ataagtatcc atcagagaca gtatctctag gcttgggcaa |
| 2521 | gagaaaagtc cacagtgata agcaactcca cctaaggcat gaatatgcgg cagagaaaac |
| 2581 | agcaatagtg aatgaatgca aaaggtgctg agaaattcca cacatgagta ttgtgatgag |
| 2641 | taaatgaata aaacatttgc aaagaccttt agagaaagag aatgggagca tatgtgagaa |
| 2701 | ataagatagt tgattatgaa tagaaggtag tgaagaaaag caagctaaga aaaaattctg |
| 2761 | tttataaaag aaggaaaaga tagtttatgt ttttagccta agtataagag tcctacagat |
| 2821 | ggactgaaaa aaatcagtct gagagtatta gtcacaatta atgaaataat tacattttat |
| 2881 | gtattgagga tgccaagatt aaaaggtgac aggtagatgt taatttccct agattgtgaa |
| 2941 | agtgatcacg acaatcacac aacaaataat taagtgactt ggtatgcttt atttaattgt |
| 3001 | agggcctgag gttttccatt ctcatttttc taaaatacaa ttttgtttct ccaaatttga |
| 3061 | cagcagaata aaaaccctac cctttcactg tgtatcatgc taagctgcat ctctactctt |
| 3121 | gatcatctgt aggtattaat cacatcactt ccatggcatg gatgttcaca tacagactct |
| 3181 | taaccctggt ttaccaggac ctctaggagt ggatccaatc tatatcttta cagttgtata |
| 3241 | gtatatgata tctcttttat ttcactcaat ttatattttc atcattgact acatatttct |
| 3301 | tatacacaac acacaattta tgaatttttt ctcaagatca ttctgagagt tgccccaccc |
| 3361 | tacctgcctt ttatagtatg cccacctcag gcagacacag agcacaatgc tggggttctc |
| 3421 | ttcacactat cactgcccca aattgtcttt ctaaatttca acttcaatgt catcttctcc |
| 3481 | atgaagacca ctgaatgaac accttttcat ccagccttaa tttcttgctc cataactact |
| 3541 | ctatcccacg atgcagtatt gtatcattaa ttattagtgt gcttgtgacc tccttatgta |
| 3601 | ttctcaatta cctgtatttg tgcaataaat tggaataatg taacttgaaa aaaaaaaa |
| SEQ ID NO: 8 = Mus musculus membrane-spanning 4-domains, subfamily A, | |
| member 2 (Ms4a2), mRNA = NCBI Reference Sequence: NM_013516.1 | |
| 1 | ggatagccca attaatgaaa aaatggacac agaaaatagg agcagagcag atcttgctct |
| 61 | cccaaatcca caagaatcct ccagtgcacc tgacattgaa ctcttggaag catctcctgc |
| 121 | caaagcagcc ccaccaaagc agacatggcg gacatttttg aagaaagagt tggagttcct |
| 181 | gggagcaaca caaattctgg ttggtttgat atgcctttgt tttggaacaa ttgtctgctc |
| 241 | cgtactctat gtttcagact ttgatgaaga agtgctttta ctttataaac taggctatcc |
| 301 | attctggggt gcagtgctgt ttgttttgtc tggatttttg tcaattatct ccgaaagaaa |
| 361 | aaacacattg tatctggtga gaggcagcct gggagcaaac attgtcagta gcatcgctgc |
| 421 | agggacgggg atcgccatgc tgatcctcaa tctgaccaat aacttcgctt atatgaacaa |
| 481 | ctgcaagaat gtaaccgaag acgacggctg ctttgtggct tctttcacca cagaactggt |
| 541 | gttgatgatg ctgtttctca ccatcctggc cttttgcagt gctgtgttgt tcactatcta |
| 601 | taggattgga caagagttag aaagtaaaaa ggtcccagat gatcgtcttt atgaagaatt |
| 661 | aaatgtgtat tcaccaattt acagtgagtt ggaagacaaa ggggaaacat cttctccagt |
| 721 | tgattcataa gaatcagggg accaggacaa tctgattcaa gtataatctt gaaagttgat |
| 781 | ctttttacaa aattctcgca aaatttctgt ttgttccaca ttctgtcagt ttttcaattg |
| 841 | gattgttctg cagatgccac tcttttagtt atgctgtatc tgatcttcta aatatctccc |
| 901 | tttttgcgga tatcattcac tccaattttc ttgttttgtg tcacaatttc acatacatct |
| 961 | tttctggaaa gtcatcaagg aataagttgg ctttattgta tgtctacttt |
| SEQ ID NO: 9 = human gamma subunit FcERI = NCBI Acc. No. NM_004106 | |
| 1 | cagaacggcc gatctccagc ccaagatgat tccagcagtg gtcttgctct tactcctttt |
| 61 | ggttgaacaa gcagcggccc tgggagagcc tcagctctgc tatatcctgg atgccatcct |
| 121 | gtttctgtat ggaattgtcc tcaccctcct ctactgtcga ctgaagatcc aagtgcgaaa |
| 181 | ggcagctata accagctatg agaaatcaga tggtgtttac acgggcctga gcaccaggaa |
| 241 | ccaggagact tacgagactc tgaagcatga gaaaccacca cagtagcttt agaatagatg |
| 301 | cggtcatatt cttctttggc ttctggttct tccagccctc atggttggca tcacatatgc |
| 361 | ctgcatgcca ttaacaccag ctggccctac ccctataatg atcctgtgtc ctaaattaat |
| 421 | atacaccagt ggttcctcct ccctgttaaa gactaatgct cagatgctgt ttacggatat |
| 481 | ttatattcta gtctcactct cttgtcccac ccttcttctc ttccccattc ccaactccag |
| 541 | ctaaaatatg ggaagggaga acccccaata aaactgccat ggactggact c |
| SEQ ID NO: 10 = murine gamma subunit FcERI = NCBI Acc. No. NM_010185 | |
| 1 | ggagggaact gtggtcaggg aactgttcgt gggcacagct gcgcagttct gtcagcgcag |
| 61 | cgcgatcacc agctcccagc gccgcagccc ccagcgcacc caggatgatc tcagccgtga |
| 121 | tcttgttctt gctccttttg gtggaacaag cagccgccct gggagagccg cagctctgct |
| 181 | atatcctgga tgctgtcctg tttttgtatg gtattgtcct taccctactc tactgtcgac |
| 241 | tcaagatcca ggtccgaaag gcagctatag ccagccgtga gaaagcagat gctgtctaca |
| 301 | cgggcctgaa cacccggagc caggagacat atgagactct gaagcatgag aaaccacccc |
| 361 | agtagcttca gaacagacgt gcttggctgc attcttttcc cacttctaat tctctccgag |
| 421 | ccctcttggt cacctctgtg ctttgaaggt tggctgacct tattcacata atgatgctag |
| 481 | ctaggctcta catcagtgta cactggcagg tccccatctc cgttaaagac ttactcactg |
| 541 | acatttctct tcttccagcc tcctttgctt catttctttt tccttccctg atcctcgact |
| 601 | ctcactaaac aatggaaagg gattatcccc caataaagct gccagagacc tgactcaaaa |
| 661 | aaaaaaaaaa aaaaaaaaaa aaa |
| SEQ ID NO; 11 = human CD23 = NCBI Acc. No. NM_002002 | |
| 1 | agtggctcta ctttcagaag aaagtgtctc tcttcctgct taaacctctg tctctgacgg |
| 61 | tccctgccaa tcgctctggt cgaccccaac acactaggag gacagacaca ggctccaaac |
| 121 | tccactaacc agagctgtga ttgtgcccgc tgagtggact gcgttgtcag ggagtgagtg |
| 181 | ctccatcatc gggagaatcc aagcaggacc gccatggagg aaggtcaata ttcagagatc |
| 241 | gaggagcttc ccaggaggcg gtgttgcagg cgtgggactc agatcgtgct gctggggctg |
| 301 | gtgaccgccg ctctgtgggc tgggctgctg actctgcttc tcctgtggca ctgggacacc |
| 361 | acacagagtc taaaacagct ggaagagagg gctgcccgga acgtctctca agtttccaag |
| 421 | aacttggaaa gccaccacgg tgaccagatg gcgcagaaat cccagtccac gcagatttca |
| 481 | caggaactgg aggaacttcg agctgaacag cagagattga aatctcagga cttggagctg |
| 541 | tcctggaacc tgaacgggct tcaagcagat ctgagcagct tcaagtccca ggaattgaac |
| 601 | gagaggaacg aagcttcaga tttgctggaa agactccggg aggaggtgac aaagctaagg |
| 661 | atggagttgc aggtgtccag cggctttgtg tgcaacacgt gccctgaaaa gtggatcaat |
| 721 | ttccaacgga agtgctacta cttcggcaag ggcaccaagc agtgggtcca cgcccggtat |
| 781 | gcctgtgacg acatggaagg gcagctggtc agcatccaca gcccggagga gcaggacttc |
| 841 | ctgaccaagc atgccagcca caccggctcc tggattggcc ttcggaactt ggacctgaag |
| 901 | ggggagttta tctgggtgga tgggagccac gtggactaca gcaactgggc tccaggggag |
| 961 | cccaccagcc ggagccaggg cgaggactgc gtgatgatgc ggggctccgg tcgctggaac |
| 1021 | gacgccttct gcgaccgtaa gctgggcgcc tgggtgtgcg accggctggc cacatgcacg |
| 1081 | ccgccagcca gcgaaggttc cgcggagtcc atgggacctg attcaagacc agaccctgac |
| 1141 | ggccgcctgc ccaccccctc tgcccctctc cactcttgag catggataca gccaggccca |
| 1201 | gagcaagacc ctgaagaccc ccaaccacgg cctaaaagcc tctttgtggc tgaaaggtcc |
| 1261 | ctgtgacatt ttctgccacc caaacggagg cagctgacac atctcccgct cctctatggc |
| 1321 | ccctgccttc ccaggagtac accccaacag caccctctcc agatgggagt gcccccaaca |
| 1381 | gcaccctctc cagatgagag tacaccccaa cagcaccctc tccagatgag agtacacccc |
| 1441 | aacagcaccc tctccagatg agagtacacc ccaacagcac cctctccaga tgcagcccca |
| 1501 | tctcctcagc accccaggac ctgagtatcc ccagctcagg tggtgagtcc tcctgtccag |
| 1561 | cctgcatcaa taaaatgggg cagtgatggc ctcccacatt tgtccccttc ttggaaaaaa |
| SEQ ID NO: 12 = murine CD23 = NCBI Acc. No. NM_013517 | |
| 1 | agaagactac tgtcttcaac acactagcct gagctacctt atccaagtgc tccacatatt |
| 61 | ccagaaggag aaggacagac ttcaagttca aatcacttcc agagctgtga gtgacaagtg |
| 121 | ccttggcagg tagtgcacgc ctcatcactg aaaggatcca aacaagactg ccatggaaga |
| 181 | aaatgaatac tcaggatact gggaacctcc tagaaagcgt tgctgctgtg caagacgtgg |
| 241 | gacacagctc atgttggtgg ggctgctgag cacagcaatg tgggceggcc tgctggccct |
| 301 | gcttcttctg tggcactggg aaacggagaa gaatctaaaa cagctgggag acactgcaat |
| 361 | tcagaatgtc tctcatgtta ccaaggactt acaaaaattc cagagtaatc aattggccca |
| 421 | gaagtcccag gttgttcaga tgtcacaaaa cttgcaagaa ctccaagctg aacagaagca |
| 481 | aatgaaagct caggactctc ggctctccca gaacctgacc ggactccagg aggatctaag |
| 541 | gaacgcccaa tcccagaact caaaactctc ccagaacctg aacagactcc aagacgatct |
| 601 | agtcaacatc aaatccctgg gcttgaatga gaagcgcaca gcctccgatt ctctagagaa |
| 661 | actccaggaa gaggtggcaa agctgtggat agagatactg atttcaaagg gaactgcatg |
| 721 | caacatatgt cccaagaact ggctccattt ccaacagaag tgctactatt ttggcaaggg |
| 781 | ctccaagcag tggatccagg ccaggttcgc ctgcagtgac ctgcaagggc gactagtcag |
| 841 | catccacagc caaaaggaac aggacttcct gatgcaacac atcaacaaga aggattcctg |
| 901 | gattggcctc caggatctca atatggaggg agagtttgta tggtcggacg ggagccctgt |
| 961 | gggttatagc aactggaatc caggggagcc caataacggg ggccagggtg aggactgtgt |
| 1021 | gatgatgcgg ggatccggcc agtggaacga cgccttctgc cgcagctact tggatgcatg |
| 1081 | ggtgtgtgag cagctggcaa catgtgagat atctgccccc ttagcctctg tgactccaac |
| 1141 | aaggcccacc ccaaaaagtg aaccctgaca aacttctgct cacactcttc tggatttctc |
| 1201 | ctctaccttt atcgtggaaa cagctgggcc ctgaggatac ccctatcagg gcccagggcc |
| 1261 | ctctctgtga ccgaaggctt tgattatgtt cccacccata ctgaagcagc tggtggatgc |
| 1321 | cagctcctgc cagctaccca gaaaccctct ccagctctcc agctaagctg gccatcccat |
| 1381 | tccatctgcc ttcctcaaac ctgggcccca gccttgctag ctccctgact acgggcatgc |
| 1441 | atgtgggcag ctgagccaac cagggagctg ctgagaacaa agatttcgaa ggcttctttt |
| 1501 | gcagtcccca cctcctatca agttccccac tttctccccc tcggcatcag agaacagggg |
| 1561 | ttccctttcc ccaggatctg ggatgagtcc tcccatcaag tttgcatcag tggtcccagc |
| 1621 | actccgaccc tccttggagg ctgcaccagg tgtgctcctg gtgcgggagg tattgaagga |
| 1681 | actctaaaca gctccagcaa ggcgagcctg gctctgtctg gtaggcctgg cccttctctc |
| 1741 | ccattccttc taccttacta aaagctgtta gagaacagtc ctaaagctag cccccaaggt |
| 1801 | ctattccctt atttggccac ttcctcctcc tgaggctgac tacaaggtcc agctatccaa |
| 1861 | gtactgaagt ctaacatcaa aagccccctt tgtctcacct aagtagcaat gcccaatcaa |
| 1921 | aatacaccat cacatcatag cccagtctaa cagaccgccc tttttctctt cataaaatta |
| 1981 | cacctgcaac caggcgtagt ggtgcaggcc tttagtccca gcacttggga ggcagagaca |
| 2041 | agcgaatttc tgagttcgag gccagcctgg tctacaaagt gagttccagg acagccaggg |
| 2101 | ctacacagag aaaccctgtc tcgaagaaag aaaaaaaaaa aaaattacac ctgcaaggtc |
| 2161 | acttgcaggc tgctgttttt ctgcctgagt cagagggcag ccacttaact tttcttccct |
| 2221 | gcttaataaa ggatctctgt g |
| SEQ ID NO: 13 = human NFKappaB = GenBank Acc. No. M58603 | |
| 1 | ggccaccgga gcggcccggc gacgatcgct gacagcttcc cctgcccttc ccgtcggtcg |
| 61 | ggccgccagc cgccgcagcc ctcggcctgc acgcagccac cggccccgct cccggagccc |
| 121 | agcgccgccg aggccgcagc cgcccggcca gtaaggcggc gccgcccgcg gccaccgcgg |
| 181 | gccctgccgt tccctccgcc gcgctgcgcc atggcgcggc gctgactggc ctggcccggc |
| 241 | cccgccgcgc tcccgctcgc cccgacccgc actcgggccc gcccgggctc cggcctgccg |
| 301 | ccgcctcttc cttctccagc cggcaggccc cgccgcttag gagggagagc ccacccgcgc |
| 361 | caggaggccg aacgcggact cgccacccgg cttcagaatg gcagaagatg atccatattt |
| 421 | gggaaggcct gaacaaatgt ttcatttgga tccttctttg actcatacaa tatttaatcc |
| 481 | agaagtattt caaccacaga tggcactgcc aacagatggc ccataccttc aaatattaga |
| 541 | gcaacctaaa cagagaggat ttcgtttccg ttatgtatgt gaaggcccat cccatggtgg |
| 601 | actacctggt gcctctagtg aaaagaacaa gaagtcttac cctcaggtca aaatctgcaa |
| 661 | ctatgtggga ccagcaaagg ttattgttca gttggtcaca aatggaaaaa atatccacct |
| 721 | gcatgcccac agcctggtgg gaaaacactg tgaggatggg atctgcactg taactgctgg |
| 781 | acccaaggac atggtggtcg gcttcgcaaa cctgggtata cttcatgtga caaagaaaaa |
| 841 | agtatttgaa acactggaag cacgaatgac agaggcgtgt ataaggggct ataatcctgg |
| 901 | actcttggtg caccctgacc ttgcctattt gcaagcagaa ggtsgagggg accggcagct |
| 961 | gggagatcgg gaaaaagagc taatccgcca agcagctctg cagcagacca aggagatgga |
| 1021 | cctcagcgtg gtgcggctca tgtttacagc ttttcttccg gatagcactg gcagcttcac |
| 1081 | aaggcgcctg gaacccgtgg tatcagacgc catctatgac agtaaagccc ccaatgcatc |
| 1141 | caacttgaaa attgtaagaa tggacaggac agctggatgt gtgactggag gggaggaaat |
| 1201 | ttatcttctt tgtgacaaag ttcagaaaga tgacatccag attcgatttt atgaagagga |
| 1261 | agaaaatggt ggagtctggg aaggatttgg agatttttcc cccacagatg ttcatagaca |
| 1321 | atttgccatt gtcttcaaaa ctccaaagta taaagatatt aatattacaa aaccagcctc |
| 1381 | tgtgtttgtc cagcttcgga ggaaatctga cttggaaact agtgaaccaa aacctttcct |
| 1441 | ctactatcct gaaatcaaag ataaagaaga agtgcagagg aaacgtcaga agctcatgcc |
| 1501 | caatttttcg gatagtttcg gcggtggtag tggtgccgga gctggaggcg gaggcatgtt |
| 1561 | tggtagtggc ggtggaggag ggggcactgg aagtacaggt ccagggtata gcttcccaca |
| 1621 | ctatggattt cctacttatg gtgggattac tttccatcct ggaactacta aatctaatgc |
| 1681 | tgggatgaag catggaacca tggacactga atctaaaaag gaccctgaag gttgtgacaa |
| 1741 | aagtgatgac aaaaacactg taaacctctt tgggaaagtt attgaaacca cagagcaaga |
| 1801 | tcaggagccc agcgaggcca ccgttgggaa tggtgaggtc actctaacgt atgcaacagg |
| 1861 | aacaaaagaa gagagtgctg gagttcagga taacctcttt ctagagaagg ctatgcagct |
| 1921 | tgcaaagagg catgccaatg cccttttcga ctacgcggtg acaggagacg tgaagatgct |
| 1981 | gctggccgtc cagcgccatc tcactgctgt gcaggatgag aatggggaca gtgtcttaca |
| 2041 | cttagcaatc atccaccttc attctcaact tgtgagggat ctactagaag tcacatctgg |
| 2101 | tttgatttct gatgacatta tcaacatgag aaatgatctg taccagacgc ccttgcactt |
| 2161 | ggcagtgatc actaagcagg aagatgtggt ggaggatttg ctgagggctg gggccgacct |
| 2221 | gagccttctg gaccgcttgg gtaactctgt tttgcaccta gctgccaaag aaggacatga |
| 2281 | taaagttctc agtatcttac tcaagcacaa aaaggcagca ctacttcttg accaccccaa |
| 2341 | cggggacggt ctgaatgcca ttcatctagc catgatgagc aatagcctgc catgtttgct |
| 2401 | gctgctggtg gccgctgggg ctgacgtcaa tgctcaggag cagaagtccg ggcgcacagc |
| 2461 | actgcacctg gctgtggagc acgacaacat ctcattggca ggctgcctgc tcctggaggg |
| 2521 | tgatgcccat gtggacagta ctacctacga tggaaccaca cccctgcata tagcagctgg |
| 2581 | gagagggtcc accaggctgg cagctcttce caaagcagca ggagcagatc ccctggtgga |
| 2641 | gaactttgag cctctctatg acctggatga ctcttgggaa aatgcaggag aggatgaagg |
| 2701 | agttgtgcct ggaaccacgc ctctagatat ggccaccagc tggcaggtat ttgacatatt |
| 2761 | aaatgggaaa ccatatgagc cagagtttac atctgatgat ttactagcac aaggagacat |
| 2821 | gaaacagctg gctgaagatg tgaagctgca gctgtataag ttactagaaa ttcctgatcc |
| 2881 | agacaaaaac tgggctactc tggcgcagaa attaggtctg gggatactta ataatgcctt |
| 2941 | ccggctgagt cctgctcctt ccaaaacact tatggacaac tatgaggtct ctgggggtac |
| 3001 | agtcagagag ctggtggagg ccctgagaca aatgggctac accgaagcaa ttgaagtgat |
| 3061 | ccaggcagcc tccagcccag tgaagaccac ctctcaggcc cactcgctgc ctctctcgcc |
| 3121 | tgcctccaca aggcagcaaa tagacgagct ccgagacagt gacagtgtct gcgacacggg |
| 3181 | cgtggagaca tccttccgca aactcagctt taccgagtct ctgaccagtg gtgcctcact |
| 3241 | gctaactctc aacaaaatgc cccatgatta tgggcaggaa ggacctctag aaggcaaaat |
| 3301 | ttagcctgct gacaatttcc cacaccgtgt aaaccaaagc cctaaaattc cactgcgttg |
| 3361 | tccacaagac agaagctgaa gtgcatccaa aggtgctcag agagccggcc cgcctgaatc |
| 3421 | attctcgatt taactcgaga ccttttcaac ttggcttcct ttcttggttc ataaatgaat |
| 3481 | tttagtttgg ttcacttaca gatagtatct agcaatcaca acactggctg agcggatgca |
| 3541 | tctggggatg aggttgctta ctaagctttg ccagctgctg ctggatcaca gctgctttct |
| 3601 | gttgtcattg ctgttgtccc tctgc |
| SEQ ID NO: 14 = murine NFKappaB = GenBank Acc. No. AY521462 | |
| 1 | atggcagacg atgatcccta cggaactggg caaatgtttc atttgaacac tgctttgact |
| 61 | cactcaatat ttaatgcaga attatattca ccagaaatac cactgtcaac agatggccca |
| 121 | taccttcaaa tattagagca accaaaacag aggggatttc gattccgcta tgtgtgtgaa |
| 181 | ggcccatcac acggagggct tccgggagcc tctagtgaga agaacaagaa atcctaccca |
| 241 | caggtcaaaa tttgcaacta tgtggggcct gcaaaggtta tcgttcagtt ggtcacaaat |
| 301 | ggaaaaaaca tccacctgca cgcccacagc ctggtgggca agcactgtga ggacggggta |
| 361 | tgcaccgtaa cagcaggacc caaggacatg gtggttggct ttgcaaacct gggaatactt |
| 421 | catgtgacta agaaaaaggt atttgaaaca ctggaagcac ggatgacaga ggcgtgtatt |
| 481 | aggggctata atcctggact tctggtgcat tctgaccttg cctatctaca agcagaaggc |
| 541 | ggaggagacc ggcaactcac agacagagag aaggagatca tccgccaggc agccgtgcag |
| 601 | cagaccaagg agatggacct gagcgtggtg cgcctcatgt tcacagcctt cctccctgac |
| 661 | agcactggca gcttcactcg gagactggag cctgtggtgt cagacgccat ctatgatagc |
| 721 | aaagccccga atgcatccaa cctgaaaatc gtgagaatgg acagaacagc aggatgtgtg |
| 781 | acgggagggg aggagattta ccttctctgt gacaaggttc agaaagatga catccagatt |
| 841 | cggttttatg aagaggaaga aaatggcgga gtttgggaag gatttgggga cttttccccc |
| 901 | acggatgttc atagacagtt tgccattgtc ttcaaaacgc caaagtataa ggatgtcaac |
| 961 | attacaaagc cagcttccgt gtttgttcag cttcggagga aatcagacct ggaaactagt |
| 1021 | gaaccgaaac cctttctcta ctaccctgaa atcaaagaca aagaggaagt gcaaaggaaa |
| 1081 | cgccagaagc ttatgccgaa cttctcggac agcttcggcg gcggcagtgg agcgggagcc |
| 1141 | ggtggtggag gcatgttcgg tagtggcggt ggcggaggga gtaccggaag ccctggccca |
| 1201 | gggtatggct actcgaacta cggatttcct ccctacggtg ggattacatt ccatcccgga |
| 1261 | gtcacgaaat ccaacgcagg ggtcacccat ggcaccataa acaccaaatt taaaaatggc |
| 1321 | cctaaagatt gtgccaagag tgatgacgag gagagtctga ctctccctga gaaggaaact |
| 1381 | gaaggtgaag ggcccagcct gcccatggcc tgcaccaaga cggaacccat cgccttggca |
| 1441 | tccaccatgg aagacaagga gcaggacatg ggatttcagg ataacctctt tctcgagaag |
| 1501 | gctctgcagc tcgccaggcg acacgccaac gcccttttcg actacgcagt gacgggggat |
| 1561 | gtgaagatgt tgctggccgt gcaacgccat ctcaccgccg tgcaggatga gaatggggac |
| 1621 | agtgtcttac acttagccat catccacctc cacgctcagc tcgtgaggga tctgctggaa |
| 1681 | gtcacatctg gtttgatctc tgatgacatc atcaacatga gaaatgacct gtatcagaca |
| 1741 | cctctgcact tggccgtgat caccaagcag gaagatgtag tagaggattt gctgagggtt |
| 1801 | ggggctgacc tgagccttct ggaccgctgg ggcaactctg tcctgcacct agctgccaaa |
| 1861 | gaaggacacg acagaatcct cagcatcctg ctcaagagca gaaaagcagc gccccttatc |
| 1921 | gaccacccca atggggaagg tctaaatgcc atccacatag ctgtgatgag caatagcctg |
| 1981 | ccatgtctgc tgctgctggt ggctgccggg gcagaagtca atgctcagga gcagaagtct |
| 2041 | gggcgcacag cgctgcacct ggccgtggag tacgacaaca tctccttggc tggctgcctg |
| 2101 | cttctggagg gtgatgccca cgtggacagt accacctatg atgggactac acctctgcat |
| 2161 | atagcggccg gaagagggtc caccagactg gcagctcttc tcaaagcagc aggagcagac |
| 2221 | cccctggtgg agaactttga gcctctctat gacctggacg actcttggga gaaggctgga |
| 2281 | gaagatgagg gagtggtgcc aggtaccaca cccctggaca tggctgccaa ctggcaggta |
| 2341 | tttgacatac taaatgggaa accgtatgag cctgtgttca catctgatga tatactacca |
| 2401 | caaggggaca tgaagcagct gacagaagac acgaggctac aactctgcaa actgctggaa |
| 2461 | attcctgatc cagacaaaaa ctgggccact ctggcacaga agttgggtct ggggatattg |
| 2521 | aacaatgcct tccggctgag tcctgctcct tctaaaactc tcatggacaa ctatgaggtc |
| 2581 | tctgggggta ccatcaaaga gctgatggag gccctgcaac agatgggcta cacagaggcc |
| 2641 | attgaagtga tccaggcagc cttccgcacc ccggcaacca cagcctccag ccccgtgacc |
| 2701 | actgctcagg tccactgtct gcctctctcg tcttcctcca cgaggcagca catagatgaa |
| 2761 | ctccgggata gtgacagcgt ctgtgacagt ggtgtggaga catccttccg caaactcagc |
| 2821 | tttacagagt ctcttactgg agacagccca ctgctatctc tgaacaaaat gccccacggt |
| 2881 | tatgggcagg aaggacctat tgaaggcaaa atttagcctg ctggccgttc ccccacactg |
| 2941 | tgtaaaccaa agccctgaca gtccattgca tcgtcccaaa ggaggaaggc aaagcgaatc |
| 3001 | caaaggtgct ggagaatcgc cggcctgcag ggtcactcga tttcattcaa ggccttccga |
| 3061 | atttggcgtc cttcttggtt ctgaaatgaa atgtagttgc cacgcacaga cggtgtctag |
| 3121 | caatcatggc gctcgctcgc tcagctgcac tctatggctc aggtgcagtg tcttgagctt |
| 3181 | tctctgctgc tactggatca catttgcttt gtgttgttac tgctgtccct ccgctgggtt |
| 3241 | cctgctgtca ttaaaaggtg tcgctgtccc cacccggtgt ccttectagc catctactgt |
| 3301 | aagttgtgca ttcaaattaa gattaaggaa aaacatattt ttaaatgagt accttgatgc |
| 3361 | gcaataaaaa aaaagacatt tcttttttta atgtggttta tctgtgattt aaaaataaaa |
| 3421 | aacacatgaa cttatcaata tttaaaacat gctacaatca gtgntgaaaa tagtattttc |
| 3481 | cccgttttat gcattttaca tttgtaaata tgttttctaa tcaatacttt aaaagaagaa |
| 3541 | tgttgaattt ataaaatgct atttactttt ttatttataa taaagtacag cacatgtgac |
| 3601 | t |

SEQ ID NO: 15 = 2. Homologous CD23: (Also from 3′ end)
gca gaa ggc gtc gtt cca
SEQ ID NO: 16 = Mouse FcERI-alpha:
cca gtg acc atc gcc cct g
SEQ ID NO: 17 = mFcERIa control
cag ggg cga tgg tca ctg g
SEQ ID NO: 18 = 5. hFcERIalpha-1:
aca gta gag tag ggg att cca tgg cag gag cca tct tct tca tgg act cc
SEQ ID NO: 19 = hFcERIalpha-2:
ttc aag gag acc tta ggt ttc tga ggg act gct aac acg cca tct gga gc
SEQ ID NO: 20 = hFcERII (CD23):
tct ctg aat att gac ctt cct cca tgg cgg tcc tgc ttg gat tct ccc ga

Various publications are cited herein, the contents of which are hereby incorporated by reference in their entireties.

What is claimed is:

1. A method of treating an inflammatory lung disorder comprising administering, to a subject in need of such treatment, an effective amount of microspheres carrying a mixture of oligonucleotides comprising at least one oligonucleotide that inhibits expression of an IgE receptor and at least one oligonucleotide that inhibits expression of NFkappaB.

2. The method of claim 1, wherein the inflammatory lung disorder is asthma.

3. The method of claim 1, where the oligonucleotides are oligodeoxynucleotides.

4. The method of claim 1, where the oligonucleotide that inhibits expression of an IgE receptor inhibits expression of FcERI.

5. The method of claim 1, where the oligonucleotide that inhibits expression of an IgE receptor inhibits expression of CD23.

6. The method of claim 1, where the oligonucleotide that inhibits expression of NfkappaB inhibits expression of p65.

7. A formulation comprising microspheres carrying a mixture of oligonucleotides comprising at least one oligonucleotide that inhibits expression of an IgE receptor and at least one oligonucleotide that inhibits expression of NfkappaB.

8. The formulation of claim 7, where the oligonucleotide that inhibits expression of an IgE receptor inhibits expression of FcERI.

9. The formulation of claim 7, where the oligonucleotide that inhibits expression of an IgE receptor inhibits expression of CD23.

10. The formulation of claim 7, where the oligonucleotide that inhibits expression of NfkappaB inhibits expression of p65.

11. The formulation of claim 7, where a single microsphere carries a mixture of oligonucleotides.

12. The formulation of claim 7, where a single microsphere carries only one species of oligonucleotide.

13. A method of reducing pulmonary inflammation in a subject in need of such treatment comprising administering, to the subject, an effective amount of microspheres carrying a mixture of oligonucleotides comprising at least one oligonucleotide that inhibits expression of an IgE receptor and at least one oligonucleotide that inhibits expression of NFkappaB.

14. The method of claim 13, wherein the pulmonary inflammation manifests as asthma.

15. The method of claim 13, where the oligonucleotides are oligodeoxynucleotides.

16. The method of claim 13, where the oligonucleotide that inhibits expression of an IgE receptor inhibits expression of FcERI.

17. The method of claim 13, where the oligonucleotide that inhibits expression of an IgE receptor inhibits expression of CD23.

18. The method of claim 13, where the oligonucleotide that inhibits expression of NFkappaB inhibits expression of p65.


SEQUENCE LISTING (20 sequences)

SEQ ID NO: 1 - DNA, 15 bp, Artificial sequence
  Synthetic oligonucleotide
ctggggactttccat

SEQ ID NO: 2 - DNA, 15 bp, Artificial sequence
  Synthetic oligonucleotide
atggaaagtccccag

SEQ ID NO: 3 - DNA, 21 bp, Artificial sequence
  Synthetic oligonucleotide
gtgtccacagcaaacagaatc

SEQ ID NO: 4 - protein, 16 aa, Artificial sequence
  Synthetic polypeptide
XXXXXXXXXXHHHHHH

SEQ ID NO: 5 - DNA, 1191 bp, Homo sapiens
agtctccagcatcctccacctgtctaccaccgagcatgggcctatatttgaagccttagatctctccagcacagtaagcaccaggagtccatgaagaagatggctcctgccatggaatcccctactctactgtgtgtagccttactgttcttcgctccagatggcgtgttagcagtccctcagaaacctaaggtctccttgaaccctccatggaatagaatatttaaaggagagaatgtgactcttacatgtaatgggaacaatttctttgaagtcagttccaccaaatggttccacaatggcagcctttcagaagagacaaattcaagtttgaatattgtgaatgccaaatttgaagacagtggagaatacaaatgtcagcaccaacaagttaatgagagtgaacctgtgtacctggaagtcttcagtgactggctgctccttcaggcctctgctgaggtggtgatggagggccagcccctcttcctcaggtgccatggttggaggaactgggatgtgtacaaggtgatctattataaggatggtgaagctctcaagtactggtatgagaaccacaacatctccattacaaatgccacagttgaagacagtggaacctactactgtacgggcaaagtgtggcagctggactatgagtctgagcccctcaacattactgtaataaaagctccgcgtgagaagtactggctacaattttttatcccattgttggtggtgattctgtttgctgtggacacaggattatttatctcaactcagcagcaggtcacatttctcttgaagattaagagaaccaggaaaggcttcagacttctgaacccacatcctaagccaaaccccaaaaacaactgatataattactcaagaaatatttgcaacattagtttttttccagcatcagcaattgctactcaattgtcaaacacagcttgcaatatacatagaaacgtctgtgctcaaggatttatagaaatgcttcattaaactgagtgaaactggttaagtggcatgtaatagtaagtgctcaattaacattggttgaataaatgagagaatgaatagattcatttattagcatttgtaaaagagatgttcaatttcaataaaataaatataaaaccatgtaacagaatgcttctgagtaaaaaaaaaaaaaaaaaaaaaaaaaaa

SEQ ID NO: 6 - DNA, 1026 bp, Mus musculus
tattttcgaagccatagctctctggtgcagttagcacctgaaggtgcaggggcgatggtcactggaaggtctgcccagctgtgcctagcactgctgttcatgtctcttgatgtcattctgacagccactgagaaatctgtactgaccttggacccaccatggattagaatatttacaggagagaaagtgaccctttcctgctatgggaacaatcaccttcaaatgaactctactactaaatggatccacaatggtaccgtctctgaggtgaactcttcacatttggtcattgtgagtgccaccgttcaagacagtggaaaatacatatgtcagaagcaaggattgtttaagagtaaacctgtgtacttgaatgtaacgcaagattggctgctccttcagacatctgctgacatggtcttagtccatggatcctttgacatcagatgccatggctggaagaactggaatgtccgcaaggtgatctactacaggaatgaccatgctttcaactacagttatgagagccccgtctccattagagaggccacactgaatgacagtggcacctaccactgcaagggctatcttaggcaggtgaaatatgaatctgacaaattcagaattgctgtagtaaaagcttacaaatgcaagtattattggctacaactaattttcccattgttggtggcgattctgtttgctgtggacacggggttattgctctcaaccgaagaacagttcaaatcagtcttggagattcagaagactggaaaatacaagaaagttgaaaccgaactcctaacctagatacatataggaactaatgtcattgctgaagaagcaattcttaacagtaattttctgcagaatcctcaatagcttctccactgtcaaaggccactcatgtgatccctagaaaagtctgttccacaggaattgcataaatgcttcattaaaccaactgcagctggttatgtaacatgcaataaatagcaaatactcaataaacactgccttaataagtta

SEQ ID NO: 7 - DNA, 3658 bp, Homo sapiens
aacccatttcaactgcctattcagagcatgcagtaagaggaaatccaccaagtctcaatataataatattctttattcctggacagctcggttaatgaaaaaatggacacagaaagtaataggagagcaaatcttgctctcccacaggagccttccagtgtgcctgcatttgaagtcttggaaatatctccccaggaagtatcttcaggcagactattgaagtcggcctcatccccaccactgcatacatggctgacagttttgaaaaaagagcaggagttcctgggggtaacacaaattctgactgctatgatatgcctttgttttggaacagttgtctgctctgtacttgatatttcacacattgagggagacattttttcatcatttaaagcaggttatccattctggggagccatatttttttctatttctggaatgttgtcaattatatctgaaaggagaaatgcaacatatctggtgagaggaagcctgggagcaaacactgccagcagcatagctgggggaacgggaattaccatcctgatcatcaacctgaagaagagcttggcctatatccacatccacagttgccagaaattttttgagaccaagtgctttatggcttccttttccactgaaattgtagtgatgatgctgtttctcaccattctgggacttggtagtgctgtgtcactcacaatctgtggagctggggaagaactcaaaggaaacaaggttccagaggatcgtgtttatgaagaattaaacatatattcagctacttacagtgagttggaagacccaggggaaatgtctcctcccattgatttataagaatcacgtgtccagaacactctgattcacagccaaggatccagaaggccaaggtcttgttaaggggctactggaaaaatttctattctctccacagcctgctggttttacattagatttattcgcctgataagaatattttgtttctgctgcttctgtccaccttaatattctccttctatttgtagatatgatagactcctatttttcttgttttatattatgaccacacacatctctgctggaaagtcaacatgtagtaagcaagatttaactgtttgattataactgtgcaaatacagaaaaaaagaaggctggctgaaagttgagttaaactttgacagtttgataatatttggttcttagggtttttttttttttttagcattcttaatagttacagttgggcatgatttgtaccatccacccatacccacacagtcacagtcacacacacatatgtattacttacactatatataacttcctatgcaaatattttaccaccagtcaataatacatttttgccaagacatgaagttttataaagatctgtataattgcctgaatcaccagcacattcactgacatgatattatttgcagattgacaagtaggaagtggggaattttattaagttactcgttgtctggggaggtaaataggttaaaaacagggaaattataagtgcagagattaacatttcacaaatgtttagtgaaacatttgtgaaaaaagaagactaaattaagacctgagctgaaataaagtgagtggaaatggaaataatggttatatctaaaacatgtagaaaaagagtaactggtagattttgttaacaaattaaagaataaagttagacaagcaactggttgactaatacattaagcgtttgagtctaagatgaaaggagaacactggttatgttgatagaatgataaaaagggtcgggcgcggaggctcacgcctgtaatcccagccctttgggaggccgaggtgggcagatcacgaagtcagtagtttgagaccagcctggccaacatagtgaaaccccgtctctactaaaaatacaaaaaaaaaattagctgggtgtggtggcagtcacctgtagtcccagctacttgggaggctgaggcaggagaatcgcttcaacctgggaggcggaggttgcagtgagccgagatcgcaccagtgcactccagccttggtgacaatgggagactccatctcaaaaaaaaaaaaaaaaaaaaaaaagataaaaagtcagaaatctgaaaagtggaggaagagtacaaatagacctaaattaagctcatttttaggctttgattttggggagacaaagggaaatgcagccatagagggcctgatgacatccaatacagagttctggtaaagataaaatttgatacaggtttggtgtcattataagagaaatcattattaaatgaagcaagttaacactctaagagaattattttgagatagaagtgaagctaagctaaacttcacatgcctataattggagggaaaaactaaggataaaatctagcctagaagatacaataattagtcataaacatgcattgtgaaactgtagagagcaggtagcccaaaatagagaaagattagataaagagaaaataagtatccatcagagacagtatctctaggcttgggcaagagaaaagtccacagtgataagcaactccacctaaggcatgaatatgcggcagagaaaacagcaatagtgaatgaatgcaaaaggtgctgagaaattccacacatgagtattgtgatgagtaaatgaataaaacatttgcaaagacctttagagaaagagaatgggagcatatgtgagaaataagatagttgattatgaatagaaggtagtgaagaaaagcaagctaagaaaaaattctgtttataaaagaaggaaaagatagtttatgtttttagcctaagtataagagtcctacagatggactgaaaaaaatcagtctgagagtattagtcacaattaatgaaataattacattttatgtattgaggatgccaagattaaaaggtgacaggtagatgttaatttccctagattgtgaaagtgatcacgacaatcacacaacaaataattaagtgacttggtatgctttatttaattgtagggcctgaggttttccattctcatttttctaaaatacaattttgtttctccaaatttgacagcagaataaaaaccctaccctttcactgtgtatcatgctaagctgcatctctactcttgatcatctgtaggtattaatcacatcacttccatggcatggatgttcacatacagactcttaaccctggtttaccaggacctctaggagtggatccaatctatatctttacagttgtatagtatatgatatctcttttatttcactcaatttatattttcatcattgactacatatttcttatacacaacacacaatttatgaattttttctcaagatcattctgagagttgccccaccctacctgccttttatagtatgcccacctcaggcagacacagagcacaatgctggggttctcttcacactatcactgccccaaattgtctttctaaatttcaacttcaatgtcatcttctccatgaagaccactgaatgaacaccttttcatccagccttaatttcttgctccataactactctatcccacgatgcagtattgtatcattaattattagtgtgcttgtgacctccttatgtattctcaattacctgtatttgtgcaataaattggaataatgtaacttgaaaaaaaaaaa

SEQ ID NO: 8 - DNA, 1010 bp, Mus musculus
ggatagcccaattaatgaaaaaatggacacagaaaataggagcagagcagatcttgctctcccaaatccacaagaatcctccagtgcacctgacattgaactcttggaagcatctcctgccaaagcagccccaccaaagcagacatggcggacatttttgaagaaagagttggagttcctgggagcaacacaaattctggttggtttgatatgcctttgttttggaacaattgtctgctccgtactctatgtttcagactttgatgaagaagtgcttttactttataaactaggctatccattctggggtgcagtgctgtttgttttgtctggatttttgtcaattatctccgaaagaaaaaacacattgtatctggtgagaggcagcctgggagcaaacattgtcagtagcatcgctgcagggacggggatcgccatgctgatcctcaatctgaccaataacttcgcttatatgaacaactgcaagaatgtaaccgaagacgacggctgctttgtggcttctttcaccacagaactggtgttgatgatgctgtttctcaccatcctggccttttgcagtgctgtgttgttcactatctataggattggacaagagttagaaagtaaaaaggtcccagatgatcgtctttatgaagaattaaatgtgtattcaccaatttacagtgagttggaagacaaaggggaaacatcttctccagttgattcataagaatcaggggaccaggacaatctgattcaagtataatcttgaaagttgatctttttacaaaattctcgcaaaatttctgtttgttccacattctgtcagtttttcaattggattgttctgcagatgccactcttttagttatgctgtatctgatcttctaaatatctccctttttgcggatatcattcactccaattttcttgttttgtgtcacaatttcacatacatcttttctggaaagtcatcaaggaataagttggctttattgtatgtctacttt

SEQ ID NO: 9 - DNA, 591 bp, Homo sapiens
cagaacggccgatctccagcccaagatgattccagcagtggtcttgctcttactccttttggttgaacaagcagcggccctgggagagcctcagctctgctatatcctggatgccatcctgtttctgtatggaattgtcctcaccctcctctactgtcgactgaagatccaagtgcgaaaggcagctataaccagctatgagaaatcagatggtgtttacacgggcctgagcaccaggaaccaggagacttacgagactctgaagcatgagaaaccaccacagtagctttagaatagatgcggtcatattcttctttggcttctggttcttccagccctcatggttggcatcacatatgcctgcatgccattaacaccagctggccctacccctataatgatcctgtgtcctaaattaatatacaccagtggttcctcctccctgttaaagactaatgctcagatgctgtttacggatatttatattctagtctcactctcttgtcccacccttcttctcttccccattcccaactccagctaaaatatgggaagggagaacccccaataaaactgccatggactggactc

SEQ ID NO: 10 - DNA, 683 bp, Mus musculus
ggagggaactgtggtcagggaactgttcgtgggcacagctgcgcagttctgtcagcgcagcgcgatcaccagctcccagcgccgcagcccccagcgcacccaggatgatctcagccgtgatcttgttcttgctccttttggtggaacaagcagccgccctgggagagccgcagctctgctatatcctggatgctgtcctgtttttgtatggtattgtccttaccctactctactgtcgactcaagatccaggtccgaaaggcagctatagccagccgtgagaaagcagatgctgtctacacgggcctgaacacccggagccaggagacatatgagactctgaagcatgagaaaccaccccagtagcttcagaacagacgtgcttggctgcattcttttcccacttctaattctctccgagccctcttggtcacctctgtgctttgaaggttggctgaccttattcacataatgatgctagctaggctctacatcagtgtacactggcaggtccccatctccgttaaagacttactcactgacatttctcttcttccagcctcctttgcttcatttctttttccttccctgatcctcgactctcactaaacaatggaaagggattatcccccaataaagctgccagagacctgactcaaaaaaaaaaaaaaaaaaaaaaaaaaa

SEQ ID NO: 11 - DNA, 1620 bp, Homo sapiens
agtggctctactttcagaagaaagtgtctctcttcctgcttaaacctctgtctctgacggtccctgccaatcgctctggtcgaccccaacacactaggaggacagacacaggctccaaactccactaaccagagctgtgattgtgcccgctgagtggactgcgttgtcagggagtgagtgctccatcatcgggagaatccaagcaggaccgccatggaggaaggtcaatattcagagatcgaggagcttcccaggaggcggtgttgcaggcgtgggactcagatcgtgctgctggggctggtgaccgccgctctgtgggctgggctgctgactctgcttctcctgtggcactgggacaccacacagagtctaaaacagctggaagagagggctgcccggaacgtctctcaagtttccaagaacttggaaagccaccacggtgaccagatggcgcagaaatcccagtccacgcagatttcacaggaactggaggaacttcgagctgaacagcagagattgaaatctcaggacttggagctgtcctggaacctgaacgggcttcaagcagatctgagcagcttcaagtcccaggaattgaacgagaggaacgaagcttcagatttgctggaaagactccgggaggaggtgacaaagctaaggatggagttgcaggtgtccagcggctttgtgtgcaacacgtgccctgaaaagtggatcaatttccaacggaagtgctactacttcggcaagggcaccaagcagtgggtccacgcccggtatgcctgtgacgacatggaagggcagctggtcagcatccacagcccggaggagcaggacttcctgaccaagcatgccagccacaccggctcctggattggccttcggaacttggacctgaagggggagtttatctgggtggatgggagccacgtggactacagcaactgggctccaggggagcccaccagccggagccagggcgaggactgcgtgatgatgcggggctccggtcgctggaacgacgccttctgcgaccgtaagctgggcgcctgggtgtgcgaccggctggccacatgcacgccgccagccagcgaaggttccgcggagtccatgggacctgattcaagaccagaccctgacggccgcctgcccaccccctctgcccctctccactcttgagcatggatacagccaggcccagagcaagaccctgaagacccccaaccacggcctaaaagcctctttgtggctgaaaggtccctgtgacattttctgccacccaaacggaggcagctgacacatctcccgctcctctatggcccctgccttcccaggagtacaccccaacagcaccctctccagatgggagtgcccccaacagcaccctctccagatgagagtacaccccaacagcaccctctccagatgagagtacaccccaacagcaccctctccagatgagagtacaccccaacagcaccctctccagatgcagccccatctcctcagcaccccaggacctgagtatccccagctcaggtggtgagtcctcctgtccagcctgcatcaataaaatggggcagtgatggcctcccacatttgtccccttcttggaaaaaa

SEQ ID NO: 12 - DNA, 2241 bp, Mus musculus
agaagactactgtcttcaacacactagcctgagctaccttatccaagtgctccacatattccagaaggagaaggacagacttcaagttcaaatcacttccagagctgtgagtgacaagtgccttggcaggtagtgcacgcctcatcactgaaaggatccaaacaagactgccatggaagaaaatgaatactcaggatactgggaacctcctagaaagcgttgctgctgtgcaagacgtgggacacagctcatgttggtggggctgctgagcacagcaatgtgggctggcctgctggccctgcttcttctgtggcactgggaaacggagaagaatctaaaacagctgggagacactgcaattcagaatgtctctcatgttaccaaggacttacaaaaattccagagtaatcaattggcccagaagtcccaggttgttcagatgtcacaaaacttgcaagaactccaagctgaacagaagcaaatgaaagctcaggactctcggctctcccagaacctgaccggactccaggaggatctaaggaacgcccaatcccagaactcaaaactctcccagaacctgaacagactccaagacgatctagtcaacatcaaatccctgggcttgaatgagaagcgcacagcctccgattctctagagaaactccaggaagaggtggcaaagctgtggatagagatactgatttcaaagggaactgcatgcaacatatgtcccaagaactggctccatttccaacagaagtgctactattttggcaagggctccaagcagtggatccaggccaggttcgcctgcagtgacctgcaagggcgactagtcagcatccacagccaaaaggaacaggacttcctgatgcaacacatcaacaagaaggattcctggattggcctccaggatctcaatatggagggagagtttgtatggtcggacgggagccctgtgggttatagcaactggaatccaggggagcccaataacgggggccagggtgaggactgtgtgatgatgcggggatccggccagtggaacgacgccttctgccgcagctacttggatgcatgggtgtgtgagcagctggcaacatgtgagatatctgcccccttagcctctgtgactccaacaaggcccaccccaaaaagtgaaccctgacaaacttctgctcacactcttctggatttctcctctacctttatcgtggaaacagctgggccctgaggatacccctatcagggcccagggccctctctgtgaccgaaggctttgattatgttcccacccatactgaagcagctggtggatgccagctcctgccagctacccagaaaccctctccagctctccagctaagctggccatcccattccatctgccttcctcaaacctgggccccagccttgctagctccctgactacgggcatgcatgtgggcagctgagccaaccagggagctgctgagaacaaagatttcgaaggcttcttttgcagtccccacctcctatcaagttccccactttctccccctcggcatcagagaacaggggttccctttccccaggatctgggatgagtcctcccatcaagtttgcatcagtggtcccagcactccgaccctccttggaggctgcaccaggtgtgctcctggtgcgggaggtattgaaggaactctaaacagctccagcaaggcgagcctggctctgtctggtaggcctggcccttctctcccattccttctaccttactaaaagctgttagagaacagtcctaaagctagcccccaaggtctattcccttatttggccacttcctcctcctgaggctgactacaaggtccagctatccaagtactgaagtctaacatcaaaagccccctttgtctcacctaagtagcaatgcccaatcaaaatacaccatcacatcatagcccagtctaacagaccgccctttttctcttcataaaattacacctgcaaccaggcgtagtggtgcaggcctttagtcccagcacttgggaggcagagacaagcgaatttctgagttcgaggccagcctggtctacaaagtgagttccaggacagccagggctacacagagaaaccctgtctcgaagaaagaaaaaaaaaaaaaattacacctgcaaggtcacttgcaggctgctgtttttctgcctgagtcagagggcagccacttaacttttcttccctgcttaataaaggatctctgtg

SEQ ID NO: 13 - DNA, 3625 bp, Homo sapiens
ggccaccggagcggcccggcgacgatcgctgacagcttcccctgcccttcccgtcggtcgggccgccagccgccgcagccctcggcctgcacgcagccaccggccccgctcccggagcccagcgccgccgaggccgcagccgcccggccagtaaggcggcgccgcccgcggccaccgcgggccctgccgttccctccgccgcgctgcgccatggcgcggcgctgactggcctggcccggccccgccgcgctcccgctcgccccgacccgcactcgggcccgcccgggctccggcctgccgccgcctcttccttctccagccggcaggccccgccgcttaggagggagagcccacccgcgccaggaggccgaacgcggactcgccacccggcttcagaatggcagaagatgatccatatttgggaaggcctgaacaaatgtttcatttggatccttctttgactcatacaatatttaatccagaagtatttcaaccacagatggcactgccaacagatggcccataccttcaaatattagagcaacctaaacagagaggatttcgtttccgttatgtatgtgaaggcccatcccatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgccggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacacgggcgtggagacatccttccgcaaactcagctttaccgagtctctgaccagtggtgcctcactgctaactctcaacaaaatgccccatgattatgggcaggaaggacctctagaaggcaaaatttagcctgctgacaatttcccacaccgtgtaaaccaaagccctaaaattccactgcgttgtccacaagacagaagctgaagtgcatccaaaggtgctcagagagccggcccgcctgaatcattctcgatttaactcgagaccttttcaacttggcttcctttcttggttcataaatgaattttagtttggttcacttacagatagtatctagcaatcacaacactggctgagcggatgcatctggggatgaggttgcttactaagctttgccagctgctgctggatcacagctgctttctgttgtcattgctgttgtccctctgc

SEQ ID NO: 14 - DNA, 3601 bp, Mus musculus
  misc_feature (3464)..(3464): n is a, c, g, or t
atggcagacgatgatccctacggaactgggcaaatgtttcatttgaacactgctttgactcactcaatatttaatgcagaattatattcaccagaaataccactgtcaacagatggcccataccttcaaatattagagcaaccaaaacagaggggatttcgattccgctatgtgtgtgaaggcccatcacacggagggcttccgggagcctctagtgagaagaacaagaaatcctacccacaggtcaaaatttgcaactatgtggggcctgcaaaggttatcgttcagttggtcacaaatggaaaaaacatccacctgcacgcccacagcctggtgggcaagcactgtgaggacggggtatgcaccgtaacagcaggacccaaggacatggtggttggctttgcaaacctgggaatacttcatgtgactaagaaaaaggtatttgaaacactggaagcacggatgacagaggcgtgtattaggggctataatcctggacttctggtgcattctgaccttgcctatctacaagcagaaggcggaggagaccggcaactcacagacagagagaaggagatcatccgccaggcagccgtgcagcagaccaaggagatggacctgagcgtggtgcgcctcatgttcacagccttcctccctgacagcactggcagcttcactcggagactggagcctgtggtgtcagacgccatctatgatagcaaagccccgaatgcatccaacctgaaaatcgtgagaatggacagaacagcaggatgtgtgacgggaggggaggagatttaccttctctgtgacaaggttcagaaagatgacatccagattcggttttatgaagaggaagaaaatggcggagtttgggaaggatttggggacttttcccccacggatgttcatagacagtttgccattgtcttcaaaacgccaaagtataaggatgtcaacattacaaagccagcttccgtgtttgttcagcttcggaggaaatcagacctggaaactagtgaaccgaaaccctttctctactaccctgaaatcaaagacaaagaggaagtgcaaaggaaacgccagaagcttatgccgaacttctcggacagcttcggcggcggcagtggagcgggagccggtggtggaggcatgttcggtagtggcggtggcggagggagtaccggaagccctggcccagggtatggctactcgaactacggatttcctccctacggtgggattacattccatcccggagtcacgaaatccaacgcaggggtcacccatggcaccataaacaccaaatttaaaaatggccctaaagattgtgccaagagtgatgacgaggagagtctgactctccctgagaaggaaactgaaggtgaagggcccagcctgcccatggcctgcaccaagacggaacccatcgccttggcatccaccatggaagacaaggagcaggacatgggatttcaggataacctctttctcgagaaggctctgcagctcgccaggcgacacgccaacgcccttttcgactacgcagtgacgggggatgtgaagatgttgctggccgtgcaacgccatctcaccgccgtgcaggatgagaatggggacagtgtcttacacttagccatcatccacctccacgctcagctcgtgagggatctgctggaagtcacatctggtttgatctctgatgacatcatcaacatgagaaatgacctgtatcagacacctctgcacttggccgtgatcaccaagcaggaagatgtagtagaggatttgctgagggttggggctgacctgagccttctggaccgctggggcaactctgtcctgcacctagctgccaaagaaggacacgacagaatcctcagcatcctgctcaagagcagaaaagcagcgccccttatcgaccaccccaatggggaaggtctaaatgccatccacatagctgtgatgagcaatagcctgccatgtctgctgctgctggtggctgccggggcagaagtcaatgctcaggagcagaagtctgggcgcacagcgctgcacctggccgtggagtacgacaacatctccttggctggctgcctgcttctggagggtgatgcccacgtggacagtaccacctatgatgggactacacctctgcatatagcggccggaagagggtccaccagactggcagctcttctcaaagcagcaggagcagaccccctggtggagaactttgagcctctctatgacctggacgactcttgggagaaggctggagaagatgagggagtggtgccaggtaccacacccctggacatggctgccaactggcaggtatttgacatactaaatgggaaaccgtatgagcctgtgttcacatctgatgatatactaccacaaggggacatgaagcagctgacagaagacacgaggctacaactctgcaaactgctggaaattcctgatccagacaaaaactgggccactctggcacagaagttgggtctggggatattgaacaatgccttccggctgagtcctgctccttctaaaactctcatggacaactatgaggtctctgggggtaccatcaaagagctgatggaggccctgcaacagatgggctacacagaggccattgaagtgatccaggcagccttccgcaccccggcaaccacagcctccagccccgtgaccactgctcaggtccactgtctgcctctctcgtcttcctccacgaggcagcacatagatgaactccgggatagtgacagcgtctgtgacagtggtgtggagacatccttccgcaaactcagctttacagagtctcttactggagacagcccactgctatctctgaacaaaatgccccacggttatgggcaggaaggacctattgaaggcaaaatttagcctgctggccgttcccccacactgtgtaaaccaaagccctgacagtccattgcatcgtcccaaaggaggaaggcaaagcgaatccaaaggtgctggagaatcgccggcctgcagggtcactcgatttcattcaaggccttccgaatttggcgtccttcttggttctgaaatgaaatgtagttgccacgcacagacggtgtctagcaatcatggcgctcgctcgctcagctgcactctatggctcaggtgcagtgtcttgagctttctctgctgctactggatcacatttgctttgtgttgttactgctgtccctccgctgggttcctgctgtcattaaaaggtgtcgctgtccccacccggtgtcctttctagccatctactgtaagttgtgcattcaaattaagattaaggaaaaacatatttttaaatgagtaccttgatgcgcaataaaaaaaaagacatttctttttttaatgtggtttatctgtgatttaaaaataaaaaacacatgaacttatcaatatttaaaacatgctacaatcagtgntgaaaatagtattttccccgttttatgcattttacatttgtaaatatgttttctaatcaatactttaaaagaagaatgttgaatttataaaatgctatttacttttttatttataataaagtacagcacatgtgact

SEQ ID NO: 15 - DNA, 18 bp, Artificial sequence
  Synthetic oligonucleotide
gcagaaggcgtcgttcca

SEQ ID NO: 16 - DNA, 19 bp, Artificial sequence
  Synthetic oligonucleotide
ccagtgaccatcgcccctg

SEQ ID NO: 17 - DNA, 19 bp, Artificial sequence
  Synthetic oligonucleotide
caggggcgatggtcactgg

SEQ ID NO: 18 - DNA, 50 bp, Artificial sequence
  Synthetic oligonucleotide
acagtagagtaggggattccatggcaggagccatcttcttcatggactcc

SEQ ID NO: 19 - DNA, 50 bp, Artificial sequence
  Synthetic oligonucleotide
ttcaaggagaccttaggtttctgagggactgctaacacgccatctggagc

SEQ ID NO: 20 - DNA, 50 bp, Artificial sequence
  Synthetic oligonucleotide
tctctgaatattgaccttcctccatggcggtcctgcttggattctcccga